(19) United States
(12) Patent Application Publication
(10) Pub. No.: US 2023/0031081 A1
(43) Pub. Date: Feb. 2, 2023

(54) LIGHT RECEIVING ELEMENT AND LIGHT RECEIVING DEVICE

(71) Applicant: SONY SEMICONDUCTOR SOLUTIONS CORPORATION, Kanagawa (JP)

(72) Inventor: Hisashi AKIYAMA, Kanagawa (JP)

(73) Assignee: SONY SEMICONDUCTOR SOLUTIONS CORPORATION, Kanagawa (JP)

(21) Appl. No.: 17/790,553

(22) PCT Filed: Dec. 11, 2020
(86) PCT No.: PCT/JP2020/046277

§ 371 Date: Jul. 1, 2022

(30) Foreign Application Priority Data
Jan. 10, 2020 (JP) 2020-002766

(51) Int. Cl. H01L 27/146; G02B 5/00; G02B 5/30; H04N 9/083

(52) U.S. Cl. CPC H01L 27/14625; H01L 27/14623; H01L 27/14685; G02B 5/008; G02B 5/3058; H04N 9/083; G02B 5/201

(57) ABSTRACT

A light receiving element of the present disclosure includes a wire grid polarizing element, wavelength selection means, and a photoelectric conversion portion from a light incident side, and the wavelength selection means includes a plurality of wavelength selection members, and the wavelength selection members constituted by a plasmon filter transmit light having different wavelengths.

### TECHNICAL FIELD

The present disclosure relates to a light receiving element and a light receiving device including a plurality of light receiving elements.

### BACKGROUND ART

An imaging element including a wire grid polarizing element, a plasmon filter, and a photoelectric conversion portion from a light incident side is well known as disclosed in, for example, JP 2018-098343A. Specifically, paragraph number [0201] of the patent publication discloses that a wire grid type polarizer 301 can be built into an imaging element 12D, and thus can be configured to be laminated on an upper layer of an interlayer film 102 laminated on a plasmon filter 121 as illustrated in FIG. 29 of the patent publication.

### CITATION LIST

### Patent Literature

[PTL 1]
JP 2018-098343A

### SUMMARY

### Technical Problem

However, the patent publication does not disclose a structure in which one imaging element can receive light having a plurality of wavelengths, or a structure in which light in a wide wavelength band can be received.

Thus, an object of the present disclosure is to provide a light receiving element having a configuration or a structure in which light having a plurality of wavelengths can be received and having a configuration or a structure in which light in a plurality of wavelengths is received, and a light receiving device including a plurality of light receiving elements.

### Solution to Problem

A light receiving element of the present disclosure for achieving the above-described object includes

a wire grid polarizing element, wavelength selection means, and a photoelectric conversion portion from a light incident side,

in which the wavelength selection means includes a plurality of wavelength selection members, and

the wavelength selection members constituted by a plasmon filter transmit light having different wavelengths.

A light receiving device of the present disclosure for achieving the above-described object includes

a plurality of light receiving element units each of which is constituted by a plurality of light receiving elements,

in which each of the light receiving elements constituting each light receiving element unit includes a wire grid polarizing element, wavelength selection means, and a photoelectric conversion portion from a light incident side,

the wavelength selection means includes a plurality of wavelength selection members, and

the wavelength selection members constituted by a plasmon filter transmit light having different wavelengths.

### BRIEF DESCRIPTION OF DRAWINGS

FIG. 1 is a schematic partial cross-sectional view of a light receiving device in Example 1 which is taken along an arrow A-A in FIGS. 2 and 3.

FIG. 2 is a schematic plan view of wire grid polarizing elements included in four light receiving elements constituting a light receiving element unit in the light receiving device in Example 1.

FIG. 3 is a schematic plan view of wavelength selection means included in the light receiving elements constituting the light receiving element unit in the light receiving device in Example 1.

FIG. 4A is a conceptual plan view illustrating the arrangement of the wire grid polarizing element included in the four light receiving elements constituting the light receiving element unit in the light receiving device in Example 1, and FIG. 4B is a conceptual plan view illustrating the arrangement of the wavelength selection means.

FIG. 5 is a conceptual plan view illustrating the arrangement of photoelectric conversion portions included in the four light receiving elements constituting the light receiving element unit in the light receiving device in Example 1.

FIG. 6 is a schematic perspective view of the wire grid polarizing element constituting the light receiving element of the present disclosure.

FIG. 7 is a schematic perspective view according to a modification example of the wire grid polarizing element.

FIGS. 8A and 8B are schematic partial cross-sectional views of the wire grid polarizing element.

FIGS. 9A and 9B are schematic partial cross-sectional views of the wire grid polarizing element.

FIG. 10 is a schematic partial cross-sectional view of a modification example of the light receiving device in Example 1 which is taken along the arrow A-A in FIGS. 2 and 3.

FIG. 11 is an equivalent circuit diagram of a photoelectric conversion portion in the light receiving device (solid-state imaging device) of Example 1.

FIG. 12 is a graph in which a relationship between an excitation wavelength in a plasmon abnormal transmission phenomenon (surface plasmon resonance) and a hole pitch P_{HL} of an adjacent hole in a second direction is obtained in a plasmon filter constituted by a plasmon resonance body constituted by a substrate in which a hole group is disposed in the second direction.

FIG. 13A is a conceptual plan view illustrating the arrangement of wire grid polarizing elements included in four light receiving elements constituting a light receiving element group in a light receiving device in Example 2, and FIG. 13B is a conceptual plan view illustrating the arrangement of wavelength selection means.

FIG. 14 is a conceptual plan view illustrating the arrangement of photoelectric conversion portions included in four light receiving elements constituting the light receiving element group in the light receiving device in Example 2.

FIG. 15 is a schematic plan view of wavelength selection means included in a light receiving element constituting a light receiving element unit in a light receiving device in Example 3.

FIG. 16 is a schematic plan view of wavelength selection means included in a light receiving element constituting a light receiving element unit in a light receiving device in Example 4.

FIGS. 17A, 17B, 17C, and 17D are schematic partial cross-sectional views of a base insulating layer and the like for illustrating a method of manufacturing a wire grid polarizing element constituting a light receiving device of the present disclosure.

FIG. 18 is a conceptual diagram of a solid-state imaging device in a case where the light receiving device of the present disclosure is applied to a solid-state imaging device.

FIG. 19 is a conceptual diagram of electronic equipment (camera) which is a solid-state imaging device to which the light receiving device of the present disclosure is applied.

FIG. 20 is a block diagram illustrating an example of a schematic configuration of a vehicle control system.

FIG. 21 is a diagram illustrating examples of positions at which a vehicle exterior information detection unit and an imaging unit are installed.

FIG. 22 is a diagram illustrating an example of a schematic configuration of an endoscopic surgery system.

FIG. 23 is a block diagram illustrating an example of functional configurations of a camera head and a CCU.

FIG. 24 is a conceptual diagram illustrating light passing through a wire grid polarizing element, and the like.

### DESCRIPTION OF EMBODIMENTS

Hereinafter, the present disclosure will be described on the basis of examples with reference to the drawings. However, the present disclosure is not limited to the examples, and various numerical values and materials in the examples are examples. The description will be given in the following order.

1. Overall description of light receiving element of the present disclosure and light receiving device of the present disclosure

2. Example 1 (light receiving element and light receiving device of the present disclosure)

3. Example 2 (modification of Example 1)

4. Example 3 (modification of Example 1 and Example 2)

5. Example 4 (another modification of Example 1 and Example 2)

6. Example 5 (application examples of light receiving devices of Example 1 to Example 4)

7. Others

<Overall Description of Light Receiving Element of the Present Disclosure and Light Receiving Device of the Present Disclosure>

It is possible to adopt a mode in which a wire grid polarizing element is configured such that a direction in which a belt-like laminated structure (to be described later) extends is consistent with a polarization direction for extinction, and a direction in which a belt-like laminated structure is repeated is consistent with a polarization direction of transmission. That is, a light reflecting layer has a function as a polarizer, attenuates a polarized wave (any one of a TE wave/an S wave and a TM wave/a P wave) having an electric field component in a direction parallel to the extension direction of the laminated structure among light incident on the wire grid polarizing element, and transmits a polarized wave (the other one of a TE wave/an S wave and a TM wave/a P wave) having an electric field component in a direction orthogonal to the extension direction of the laminated structure (the repetition direction of the belt-like laminated structure). That is, the extension direction of the laminated structure is a light absorption axis of the wire grid polarizing element, and the direction orthogonal to the extension direction of the laminated structure is a light transmission axis of the wire grid polarizing element. The extension direction (the light absorption axis of the wire grid polarizing element) of the belt-like laminated structure (that is, constituting a line portion of a line-and-space structure) may be referred to as a “first direction” for convenience, and the repetition direction (the direction orthogonal to the extension direction of the belt-like laminated structure, and the light transmission axis of the wire grid polarizing element) of the belt-like laminated structure (line portion) may be referred to as a “second direction” for convenience.

An angle formed by an angle α to be described later and the second direction can be substantially set to be any angle, and can be set to 0 degrees or 90 degrees. However, the present disclosure is not limited thereto.

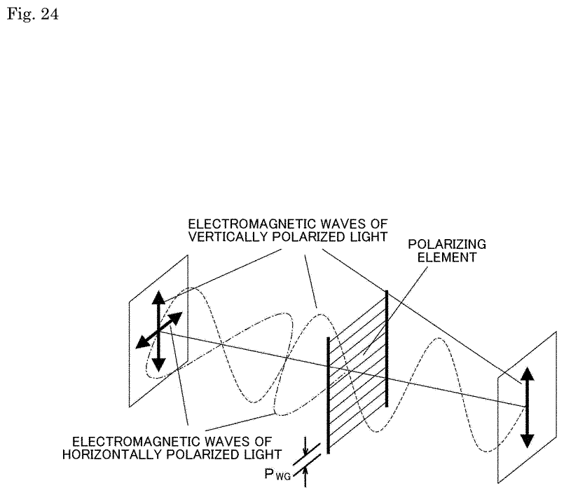

As illustrated in a conceptual diagram of FIG. 24, in a case where a formation pitch P_{WG} of the wire grid polarizing element is significantly smaller than a wavelength λ₀ of an incident electromagnetic wave, electromagnetic waves vibrating in a plane parallel to the extension direction (first direction) of the wire grid polarizing element are selectively reflected and absorbed by the wire grid polarizing element. Here, a distance between line portions (a distance or a length of a space portion in the second direction) is set to be the formation pitch P_{WG} of the wire grid polarizing element. Then, as illustrated in FIG. 24, electromagnetic waves (light) reaching the wire grid polarizing element include vertically polarized components and horizontally polarized components, but in electromagnetic waves having passed through the wire grid polarizing element, a vertically polarized component is dominant linearly polarized light. Here, considering a visible light wavelength band, in a case where when the formation pitch P_{WG} of the wire grid polarizing element is significantly smaller than an effective wavelength λ_{eff} of an electromagnetic wave incident on the wire grid polarizing element, a polarization component biased to a plane parallel to the first direction is reflected or absorbed by the surface of the wire grid polarizing element. On the other hand, when an electromagnetic wave having a polarization component biased to a plane parallel to the second direction is incident on the wire grid polarizing element, an electric field having propagated through the surface of the wire grid polarizing element is transmitted (emitted) with the same wavelength and polarization direction as an incidence wavelength from the rear surface of the wire grid polarizing element. Here, when an average refractive index obtained on the basis of a material provided in the space portion is set to be nₐᵥₑ, an effective wavelength λ_{eff} is represented by (λ₀/nₐᵥₑ). The average refractive index nₐᵥₑ is a value obtained by summing products of a refractive index and pieces of material provided in the space portion and dividing the value by the volume of the space portion. In a case where the value of the wavelength λ₀ is set to be fixed, the value of the effective wavelength λ_{eff} increases as the value of nₐᵥₑ decreases, and thus the value of the formation pitch P_{WG} can be increased. In addition, an increase in the value of nₐᵥₑ results in a decrease in a transmittance in the wire grid polarizing element and a decrease in an extinction ratio.

In the light receiving element of the present disclosure or the light receiving element constituting the light receiving device of the present disclosure (hereinafter, these light receiving elements may be collectively referred to simply as “the light receiving element and the like of the present disclosure”), it is possible to adopt a mode in which

each wavelength selection member is constituted by a periodic structure, and a periodic structure is constituted by a substrate and a plurality of holes provided in the substrate. In such a mode, it is possible to adopt a mode in which a polarization direction (second direction) of transmission performed by a wire grid polarizing element and an arrangement direction of the plurality of holes are parallel to each other. That is, it is possible to adopt a mode in which the arrangement direction of the plurality of holes and the second direction are parallel to each other. In such a mode, it is possible to adopt a mode in which a hole group is constituted by the plurality of holes arranged in the second direction and constituting each wavelength selection member, and a plurality of hole groups are arranged in a first direction. Further, it is possible to adopt a mode in which the hole groups constituting each wavelength selection member are alternately arranged in the first direction.

Alternatively, in the light receiving element and the like of the present disclosure, it is possible to adopt a mode in which

each wavelength selection member is constituted by a periodic structure, and a periodic structure is constituted by a base layer, dots provided on the base layer, and a dielectric material provided on the base layer and filled between the dots. In such a mode, it is possible to adopt a mode in which a polarization direction (second direction) of transmission performed by the wire grid polarizing element and an arrangement direction of a plurality of dots are parallel to each other. That is, it is possible to adopt a mode in which the arrangement direction of the plurality of dots is parallel to the second direction. In such a mode, it is possible to adopt a mode in which a dot group is constituted by the plurality of dots arranged in the second direction and constituting each wavelength selection member, and a plurality of dot groups are arranged in a first direction. Further, it is possible to adopt a mode in which the dot groups constituting each wavelength selection member are alternately arranged in the first direction.

Alternatively, in the light receiving element and the like of the present disclosure, it is possible to adopt a configuration in which

each wavelength selection member is constituted by a periodic structure, and the periodic structure is constituted by a base layer, and a plurality of belt-like (lattice-like) conductive material layers provided on the base layer. In such a mode, it is possible to adopt a mode in which a polarization direction (second direction) of transmission performed by a wire grid polarizing element and a repetition direction of the belt-like conductive material layers are parallel to each other. In other words, it is possible to adopt a mode in which an extension direction of the belt-like conductive material layers is a first direction, and it is possible to adopt a mode in which a repetition direction of the belt-like conductive material layers is a second direction. In such a mode, it is possible to adopt a mode in which a conductive material layer group is constituted by the plurality of belt-like conductive material layers arranged in the second direction and constituting each wavelength selection member, it is possible to adopt a mode in which a plurality of conductive material layer groups are arranged in the first direction, and it is possible to adopt a mode in which the conductive material layer groups constituting each wavelength selection member are alternately arranged in the first direction.

It is possible to adopt a mode in which the light receiving device of the present disclosure is constituted by a plurality of light receiving element groups arranged two-dimensionally,

one light receiving element group is constituted by four light receiving element units arranged in a 2×2 array,

a first light receiving element unit includes first wavelength selection means that transmits light in a first wavelength range,

a second light receiving element unit includes second wavelength selection means that transmits light in a second wavelength range,

a third light receiving element unit includes third wavelength selection means that transmits light in a third wavelength range, and

a fourth light receiving element unit includes fourth wavelength selection means that transmits light in a fourth wavelength range.

Specifically, one light receiving element group is constituted by, for example, four light receiving element units arranged in a Bayer array, and examples of light to be received may include red light as light in a first wavelength range, green light as light in a second wavelength range and light in a third wavelength range, and blue light as light in a fourth wavelength range. Alternatively, examples thereof may include red light as light in a first wavelength range, green light as light in a second wavelength range, blue light as light in a third wavelength range, and infrared light as light in a fourth wavelength range.

It is possible to adopt a mode in which the first light receiving element unit, the second light receiving element unit, the third light receiving element unit, and the fourth light receiving element unit are constituted by a first light receiving element, a second light receiving element, a third light receiving element, and a fourth light receiving element, respectively. That is, it is possible to adopt a mode in which each light receiving element unit is constituted by four light receiving elements arranged in a 2×2 array,

a polarization direction of transmission performed by a wire grid polarizing element constituting the first light receiving element of the first light receiving element unit, the first light receiving element of the second light receiving element unit, the first light receiving element of the third light receiving element unit, and the first light receiving element of the fourth light receiving element unit is at α degrees,

a polarization direction of transmission performed by a wire grid polarizing element constituting the second light receiving element of the first light receiving element unit, the second light receiving element of the second light receiving element unit, the second light receiving element of the third light receiving element unit, and the second light receiving element of the fourth light receiving element unit is at (α+45) degrees,

a polarization direction of transmission performed by a wire grid polarizing element constituting the third light receiving element of the first light receiving element unit, the third light receiving element of the second light receiving element unit, the third light receiving element of the third light receiving element unit, and the third light receiving element of the fourth light receiving element unit is at (α+90) degrees, and

a polarization direction of transmission performed by a wire grid polarizing element constituting the fourth light receiving element of the first light receiving element unit, the fourth light receiving element of the second light receiving element unit, the fourth light receiving element of the third light receiving element unit, and the fourth light receiving element of the fourth light receiving element unit is at (α+135) degrees.

In the light receiving device of the present disclosure including the above-described various preferred modes, the plurality of light receiving elements are arranged in a two-dimensional matrix. However, for convenience, one arrangement direction of the light receiving element is referred to as an “x₀ direction”, and the other arrangement direction is referred to as a “y₀ direction”. It is preferable that the x₀ direction and the y₀ direction be orthogonal to each other. The x₀ direction is a so-called row direction or a so-called column direction, and the y₀ direction is a column direction or a row direction.

In the light receiving element and the like of the present disclosure including the above-described preferred configurations, it is possible to adopt a mode in which the wire grid polarizing element is configured such that a plurality of laminated structures each including at least a belt-like light reflecting layer and a light absorbing layer (the light absorbing layer is positioned on a light incident side) are separately arranged in parallel (that is, a configuration including a line-and-space structure). Alternatively, it is possible to adopt a mode in which the wire grid polarizing element is configured such that a plurality of laminated structures each including a belt-like light reflecting layer, an insulating film, and a light absorbing layer (the light absorbing layer is positioned on a light incident side) are separately arranged in parallel. Further, in this case, it is also possible to adopt a configuration in which the light reflecting layer and the light absorbing layer in the laminated structure are separated from each other by an insulating film (that is, a configuration in which the insulating film is formed on the entire top surface of the light reflecting layer, and the light absorbing layer is formed on the entire top surface of the insulating film), and it is also possible to adopt a configuration in which a portion of the insulating film is notched, and the light reflecting layer and the light absorbing layer are in contact with each other in the notch portion of the insulating film. In these cases, it is possible to adopt a mode in which the light reflecting layer is formed of a first conductive material, and the light absorbing layer is formed of a second conductive material. With such a configuration, the entire region of the light absorbing layer and the light reflecting layer can be electrically connected to a region having an appropriate potential in the light receiving device. As a result, it is possible to reliably avoid the occurrence of a problem that the wire grid polarizing element or a photoelectric conversion portion is damaged due to charging of the wire grid polarizing element and the occurrence of a kind of discharge at the time of forming the wire grid polarizing element. Alternatively, it is possible to adopt a configuration in which a wire grid polarizing element is configured such that an insulating film is omitted, and a light absorbing layer and a light reflecting layer are laminated from a light incident side.

These wire grid polarizing elements can be manufactured on the basis of, for example, the following steps including:

(A) a step of forming, for example, a photoelectric conversion portion and then providing a light reflecting layer formation layer, which is formed of a first conductive material and is electrically connected to a substrate or a photoelectric conversion portion, on the photoelectric conversion portion,

(B) a step of providing an insulating film formation layer on a light reflecting layer formation layer and providing a light absorbing layer formation layer, which is formed of a second conductive material and of which at least a portion is in contact with the light reflecting layer formation layer, on the insulating film formation layer, and
(C) a step of patterning the light absorbing layer formation layer, the insulating film formation layer, and the light reflecting layer formation layer to obtain a wire grid polarizing element configured such that a plurality of lines portions of a belt-like light reflecting layer, an insulating film, and a light absorbing layer are separately arranged in parallel.

Further, it is possible to adopt a mode in which in the step (B), the light absorbing layer formation layer formed of the second conductive material is provided in a state where the light reflecting layer formation layer is set to be at a predetermined potential through the substrate or the photoelectric conversion portion, and

in the step (C), the light absorbing layer formation layer, the insulating film formation layer, and the light reflecting layer formation layer are patterned in a state where the light reflecting layer formation layer is set to be at a predetermined potential through the substrate or the photoelectric conversion portion.

In addition, it is possible to adopt a configuration in which a base film is formed under the light reflecting layer, and thus it is possible to improve roughness of the light reflecting layer formation layer and the light reflecting layer. Examples of a material for forming the base film (barrier metal layer) may include Ti, TiN, and a laminated structure of Ti/TiN.

Light is incident from the light absorbing layer constituting the wire grid polarizing element. The wire grid polarizing element attenuates a polarized wave (any one of a TE wave/an S wave and a TM wave/a P wave) having an electric field component parallel to a first direction by using four actions of transmission, reflection, and interference of light, and selective light absorption of polarized waves based on optical anisotropy, and transmits a polarized wave (the other one of a TE wave/an S wave and a TM wave/a P wave) having an electric field component parallel to a second direction. That is, one polarized wave (for example, a TE wave) is attenuated by selective light absorption of a polarized wave due to optical anisotropy of the light absorbing layer. The belt-like light reflecting layer functions as a polarizer, and one polarized wave (for example, a TE wave) having passed through the light absorbing layer and the insulating film is reflected by the light reflecting layer. In this case, when the insulating film is configured such that the phase of one polarized wave (for example, a TE wave) having passed through the light absorbing layer and reflected by the light reflecting layer deviates by a half wavelength, one polarized wave (for example, a TE wave) reflected by the light reflecting layer is canceled out and is attenuated by interference with one polarized wave (for example, a TE wave) reflected by the light absorbing layer. As described above, one polarized wave (for example, a TE wave) can be selectively attenuated. However, as described above, it is possible to realize an improvement in contrast even when the thickness of the insulating film is not optimized. Thus, in practice, the thickness of the insulating film may be determined on the basis of a balance between desired polarization characteristics and the actual manufacturing process.

In the following description, a laminated structure constituting a wire grid polarizing element provided above a photoelectric conversion portion may be referred to as a “first laminated structure” for convenience, and a laminated structure surrounding the first laminated structure may be referred to as a “second laminated structure” for convenience. The second laminated structure connects a wire grid polarizing element (first laminated structure) constituting a certain light receiving element and a wire grid polarizing element (first laminated structure) constituting a light receiving element adjacent to the certain light receiving element. The second laminated structure can be constituted by a laminated structure (that is, a second laminated structure constituted by at least a light reflecting layer and a light absorbing layer, for example, constituted by a light reflecting layer, an insulating film, and a light absorbing layer, and a so-called solid film structure which is not provided with a line-and-space structure) having the same configuration as that of the laminated structure constituting the wire grid polarizing element. The second laminated structure may be provided with a line-and-space structure as in the wire grid polarizing element as long as the second laminated structure does not function as a wire grid polarizing element. That is, the second laminated structure may have a structure in which a formation pitch P_{WG} of the wire grid is sufficiently larger than an effective wavelength of incident electromagnetic waves. A frame portion to be described below may also be constituted by a second laminated structure. In some cases, a frame portion may be constituted by a first laminated structure. It is preferable that the frame portion be connected to a line portion of a wire grid polarizing element. The frame portion can also function as a light shielding portion.

A light reflecting layer (light reflecting layer formation layer) can also be formed of a metal material, an alloy material, or a semiconductor material, and a light absorbing layer can be formed of a metal material, an alloy material, or a semiconductor material.

Specifically, examples of an inorganic material for forming the light reflecting layer (light reflecting layer formation layer) may include metal materials such as aluminum (Al), silver (Ag), gold (Au), copper (Cu), platinum (Pt), molybdenum (Mo), chromium (Cr), titanium (Ti), nickel (Ni), tungsten (W), iron (Fe), silicon (Si), germanium (Ge), and tellurium (Te), alloy materials containing these metals, and a semiconductor material.

Examples of a material for forming the light absorbing layer (or a light absorbing layer formation layer) may include a metal material, an alloy material, and a semiconductor material having an extinction coefficient k, which is not zero, that is, having a light absorption action. Specifically, examples thereof include metal materials such as aluminum (Al), silver (Ag), gold (Au), copper (Cu), molybdenum (Mo), chromium (Cr), titanium (Ti), nickel (Ni), tungsten (W), iron (Fe), silicon (Si), germanium (Ge), tellurium (Te), and tin (Sn), alloy materials containing these metals, and a semiconductor material. In addition, examples thereof also include silicide materials such as FeSi₂ (particularly, β-FeSi₂), MgSi₂, NiSi₂, BaSi₂, CrSi₂, and CoSi₂. In particular, by using aluminum or an alloy thereof, or a semiconductor material containing β-FeSi₂, germanium, or tellurium as a material for forming the light absorbing layer (light absorbing layer formation layer), high contrast (high extinction ratio) can be obtained in a visible light region. In addition, in order to provide polarization characteristics to a wavelength band other than a visible light region, for example, an infrared region, it is preferable to use silver (Ag), copper (Cu), gold (Au), or the like as a material for forming the light absorbing layer (light absorbing layer formation layer). This is because resonance wavelengths of these metals are in the vicinity of an infrared region.

The light reflecting layer formation layer and the light absorbing layer formation layer can be formed on the basis of known methods such as various chemical vapor deposition methods (CVD method), a coating method, various physical vapor deposition methods (PVD method) including a sputtering method and a vacuum vapor deposition method, a sol-gel method, a plating method, an MOCVD method, and an MBE method. In addition, examples of a patterning method for the light reflecting layer formation layer and the light absorbing layer formation layer may include a combination of lithography technology and etching technology (for example, anisotropic dry etching technology using carbon tetrafluoride gas, sulfur hexafluoride gas, trifluoromethane gas, xenon difluoride gas, or the like, or physical etching technology), so-called lift-off technology, and so-called self-align double patterning technology using a sidewall as a mask. Examples of the lithography technology may include photolithography technology (lithography technology using g rays and i rays of a high-pressure mercury lamp, a KrF excimer laser, an ArF excimer laser, EUV, or the like as a light source, and immersion lithography technology, electron beam lithography technology, and X-ray lithography thereof). Alternatively, the light reflecting layer and the light absorbing layer can also be formed on the basis of microfabrication technology using an ultra-short pulse laser such as a femtosecond laser, and a nanoimprint method.

Examples of materials for forming an insulating film (or an insulating film formation layer), an interlayer insulating layer, and a base insulating layer may include an insulating material which is transparent to incident light and does not have light absorption characteristics. Specifically, examples thereof may include an SiOX-based material (a material for forming a silicon-based oxide film) such as silicon oxide (SiO₂), non-doped silicate glass (NSG), boron phosphorus silicate glass (BPSG), PSG, BSG, PbSG, AsSG, SbSG, or spin-on glass (SOG), SiN, silicon oxynitride (SiON), SiOC, SiOF, SiCN, low dielectric constant insulating materials (for example, fluorocarbons, a cycloperfluorocarbon polymer, benzocyclobutene, cyclic fluororesins, polytetrafluoroethylene, amorphous tetrafluoroethylene, polyaryl ether, fluoride aryl ether, fluoride polyimide, organic SOG, parylene, fluoride fullerene, and amorphous carbon), polyimide-based resins, fluororesins, Silk (a trademark of The Dow Chemical Co., a coating-type low-dielectric-constant interlayer insulating film material), and Flare (a trademark of Honeywell Electronic Materials Co., a polyallyl ether (PAE)-based material), and these materials can be used alone or in combination as appropriate. Alternatively, examples thereof may include polymethyl methacrylate (PMMA); polyvinyl phenol (PVP); polyvinyl alcohol (PVA); polyimide; polycarbonate (PC); polyethylene terephthalate (PET); polystyrene; silanol derivatives (silane coupling agents) such as N-2(aminoethyl)3-aminopropyltrimethoxysilane (AEAPTMS), 3-mercaptopropyltrimethoxysilane (MPTMS), octadecyltrimethoxysilane (OTS); novolac phenolic resin; fluorine resins; an organic insulating material (organic polymer) exemplified by a linear hydrocarbon having a functional group capable of being coupled to a control electrode at one end such as octadecanthiol, dodecyl isocyanate, or the like, and combinations thereof can also be used. The insulating film formation layer can be formed on the basis of known methods such as various CVD methods, a coating method, various PVD methods including a sputtering method and a vacuum vapor deposition method, various printing methods, such as a screen printing method, and a sol-gel method. The insulating film functions as a base layer of the light absorbing layer, and is formed for the purpose of adjusting the phases of polarized light reflected by the light absorbing layer and polarized light having passed through the light absorbing layer and reflected by the light reflecting layer, improving an extinction ratio and a transmittance by an interference effect, and reducing a reflectance. Thus, it is preferable that the insulating film have such a thickness that the phase thereof deviates by half a wavelength in one round trip. However, the light absorbing layer has a light absorption effect, and thus reflected light is absorbed. Thus, it is possible to realize an improvement in an extinction ratio even when the thickness of the insulating film is not optimized. Therefore, in practice, the thickness of the insulating film may be determined on the basis of a balance between desired polarization characteristics and the actual manufacturing process, and the thickness can be, for example, 1×10⁻⁹ m to 1×10⁻⁷ m, and more preferably, 1×10⁻⁸ m to 8×10⁻⁸ m. In addition, a refractive index of the insulating film is larger than 1.0 and is not limited, but is preferably 2.5 or less.

A space portion of the wire grid polarizing element may also be in the form of a void (that is, the space portion is filled with at least air). In this manner, the space portion of the wire grid polarizing element is configured as a void, and thus the value of an average refractive index nₐᵥₑ can be reduced. As a result, it is possible to achieve an improvement in a transmittance and an improvement in an extinction ratio in the wire grid polarizing element. In addition, the value of a formation pitch P_{WG} can be increased, and thus it is possible to achieve an improvement in a manufacturing yield of the wire grid polarizing element.

It is also possible to adopt a mode in which a protection film is formed on the wire grid polarizing element, and thus it is possible to provide a light receiving element and a light receiving device having high reliability. Further, it is possible to improve reliability such as an improvement in moisture resistance of the wire grid polarizing element by providing the protection film. The thickness of the protection film may be set to be a thickness in a range that does not affect polarization characteristics. A reflectance for incident light also changes depending on an optical thickness of the protection film (a refractive index×a film thickness of the protection film), and thus a material of thickness of the protection film may be determined in consideration of the change in reflectance. The thickness can be set to, for example, 15 nm or less, or can be set to be, for example, ¼ or less of a distance between laminated structures. As a material for forming the protection film, a material having a refractive index of 2 or less, and a material having an extinction coefficient close to zero are preferably used. Examples of the material may include insulating materials such as SiO₂, SiON, SiN, SiC, SiOC, and SiCN containing TEOS-SiO₂, and metal oxides such as aluminum oxide (AlOX), hafnium oxide (HfOₓ), zirconium oxide (ZrOₓ), and tantalum pentoxide (TaOₓ). Alternatively, examples thereof may include perfluorodecyltrichlorosilane and octadecyltrichlorosilane. The protection film can be formed by a known process such as various CVD methods, a coating method, various PVD methods including a sputtering method and a vacuum deposition method, and a sol-gel method, but it is more preferable to adopt a so-called monoatomic layer deposition method (an ALD method, an atomic layer deposition method) and an HDP-CVD method (high density plasma chemical vapor deposition method). A thin protection film can be conformally formed on a wire grid polarizing element by adopting an ALD method or an HDP-CVD method. The protection film may be formed on the entire surface of the wire grid polarizing element, but can be configured to be formed on only a side surface of the wire grid polarizing element and not to be formed on a base insulating layer positioned between wire grid polarizing elements. In this manner, by forming the protection film so as to cover a side surface which is an exposed portion of a metal material for forming the wire grid polarizing element, or the like, it is possible to block moisture and organic substances in the air and to reliably suppress the occurrence of problems such as corrosion and abnormal precipitation of a metal material for forming the wire grid polarizing element. It is possible to achieve an improvement in long-term reliability of a light receiving element and provide a light receiving element equipped with a wire grid polarizing element having higher reliability in an on-chip manner.

In a case where a protection film is formed on a wire grid polarizing element, it is also adopt a mode in which a second protection film is formed between the wire grid polarizing element and the protection film, and

when a refractive index of a material for forming the protection film is set to be n₁′, and a refractive index of a material for forming the second protection film is set to be n₂′, a relationship of n₁′>n₂′ is satisfied. By satisfying the relationship of n₁′>n₂′, the value of an average refractive index nₐᵥₑ can be reliably reduced. Here, it is preferable that the protection film be formed of SiN, and the second protection film be formed of SiO₂ or SiON.

Further, it is possible to adopt a mode in which a third protection film is formed on a side surface of a line portion facing at least a space portion of the wire grid polarizing element. That is, the space portion is filled with air, and the third protection film is provided in the space portion. Here, as a material for forming the third protection film, a material having a refractive index of 2 or less and an extinction coefficient close to zero is preferably used. Examples of the material may include insulating materials such as SiO₂, SiON, SiN, SiC, SiOC, and SiCN containing TEOS-SiO₂, and metal oxides such as aluminum oxide (AlOX), hafnium oxide (HfOₓ), zirconium oxide (ZrOₓ), and tantalum pentoxide (TaOₓ). Alternatively, examples thereof may include perfluorodecyltrichlorosilane and octadecyltrichlorosilane. The third protection film can be formed by a known process such as various CVD methods, a coating method, various PVD methods including a sputtering method and a vacuum deposition method, and a sol-gel method, but it is more preferable to adopt a so-called monoatomic layer deposition method (an ALD method, an atomic layer deposition method) and an HDP-CVD method (high density plasma chemical vapor deposition method). A thin third protection film can be conformally formed on a wire grid polarizing element by adopting an ALD method. However, it is further preferable to adopt an HDP-CVD method from the viewpoint of forming a thinner third protection film on the side surface of the line portion. Alternatively, when the space portion is filled with the material for forming the third protection film and the third protection film is provided with gaps, pores, voids, or the like, a refractive index of the entire third protection film can be reduced.

When a metal material or an alloy material for forming the wire grid polarizing element (hereinafter, may be referred to as a “metal material and the like”) comes into contact with the outside air, there is a concern that corrosion resistance of the metal material and the like may deteriorate due to the adhesion of moisture or organic substances from the outside air, and the long-term reliability of a photoelectric conversion portion may deteriorate. In particular, when moisture adheres to line portions (laminated structure) of a metal material and the like, an insulation material, and a metal material and the like, CO₂ and O₂ are dissolved in the moisture, which acts as an electrolytic solution, and there is a concern that a local battery may be formed between two types of metals. When such a phenomenon occurs, a reduction reaction such as generation of hydrogen proceeds on a cathode side (positive electrode side), and an oxidation reaction proceeds on an anode side (negative electrode side), which results in the occurrence of abnormal precipitation of metal materials and the like and a shape change of the wire grid polarizing element. As a result, there is a concern that the originally expected performance of the wire grid polarizing element and the photoelectric conversion portion may be impaired. For example, in a case where aluminum (Al) is used as the light reflecting layer, there is a concern that abnormal precipitation of aluminum as shown in the following reaction formula may occur. However, when the protection film and the third protection film are formed, it is possible to reliably avoid the occurrence of such a problem.

Al→Al³⁺+3e⁻

Al³⁺+3OH⁻→Al(OH)₃

In the wire grid polarizing element, the length of the light reflecting layer in a first direction can be set to be the same as the length of the photoelectric conversion region, which is a region of the light receiving element for substantially performing photoelectric conversion, in the first direction, can also be set to be the same as the length of the light receiving element, and can also be set to be an integral multiple of the length of the light receiving element in the first direction, but the present disclosure is not limited thereto.

A case where a hole penetrates a substrate and functions as a waveguide is assumed. In this case, the waveguide includes a cut-off frequency (cut-off wavelength λ_{cut-off}) which is determined depending on the diameter of the hole. Light having a frequency being a cut-off frequency or less (a wavelength being a cut-off wavelength λ_{cut-off} or more) has a property of not propagating through the waveguide. The cut-off wavelength λ_{cut-off} depends on the diameter of the hole, and the cut-off wavelength λ_{cut-off} decreases as the diameter becomes smaller.

On the other hand, when light having a wavelength λ₀ is incident on a substrate in which holes are periodically formed in a period P_{HL} shorter than a desired wavelength λ₀ (>λ_{cut-off}), a phenomenon in which light having such a wavelength λ₀ passes through the substrate occurs. Such a phenomenon is referred to as a plasmon abnormal transmission phenomenon (surface plasmon resonance), and a filter that controls the transmission of light by applying such a phenomenon is referred to as a plasmon filter. Such a phenomenon occurs due to excitation of surface plasmon which occurs at a boundary between the substrate and an interlayer film formed thereon (an interlayer film covering the substrate) (for details, see JP 2018-098343A).

The plasmon filter in the light receiving element and the like of the present disclosure is constituted by a plasmon resonance body constituted by, for example, a substrate in which a hole group is disposed in the second direction as described above.

A wavelength band (a resonance wavelength of plasmon, will be referred to as a “transmission band” below) of light transmitted by the plasmon filter depends on the material and film thickness of the substrate, the material and film thickness of the interlayer film, a pattern period of a hole array (for example, a diameter D₀ of a hole and a pitch P_{HL} of an adjacent hole in the second direction), and the like. In addition, the pitch of the adjacent hole in the second direction may be referred to as a “hole pitch P_{HL}” below. In a case where the materials and film thicknesses of the substrate and the interlayer film are determined, a transmission band of the plasmon filter depends on a pattern period of a hole array, particularly, a hole pitch P_{HL}. That is, as the hole pitch P_{HL} becomes smaller, a transmission band of the plasmon filter is more shifted to a short wavelength side, and as the hole pitch P_{HL} becomes larger, a transmission band of the plasmon filter is more shifted to a long wavelength side. In addition, a light transmittance of the plasmon filter mainly depends on the diameter D₀ of the hole. As the diameter D₀ becomes larger, a light transmittance increases, but color mixture is more likely to occur. The diameter D₀ is preferably 50% to 60% of the hole pitch P_{HL}.

Examples of a material for forming the substrate may include a conductor thin film, for example, a thin film formed of a material for forming the above-described light reflecting layer, and specifically, aluminum (Al), silver (Ag), gold (Au), or copper (Cu). In addition, as a material for forming the interlayer film, it is preferable to use a material having a low dielectric constant. Examples of the material may include a material having a low dielectric constant among the above-described materials for forming the insulating film, and specifically, SiO₂, a Low-K material, and the like.

Further, in the plasmon resonance body, all of the holes do not need to penetrate the substrate, and the plasmon resonance body functions as a plasmon filter even when some of the holes are configured as non-through holes that do not penetrate the substrate. That is, holes configured as through holes and holes configured as non-through holes may be periodically disposed. Further, the plasmon filter may be constituted by a single-layer plasmon resonance body, or may be constituted by, for example, a plasmon resonance body of a plurality of laminated layers (for example, two layers).

Alternatively, the plasmon filter in the light receiving element and the like of the present disclosure is constituted by, for example, a plasmon resonance body in which a dot group is disposed in the second direction as described above. The plasmon filter functions as a filter that absorbs light in a predetermined wavelength band. A wavelength band of light absorbed by the plasmon filter (hereinafter, referred to as an “absorption band”) depends on a pitch P_{DT} of an adjacent dot in the second direction. In addition, the pitch of the adjacent dot in the second direction may be referred to as a “dot pitch P_{DT}” below. In addition, the diameter of a dot is adjusted in accordance with the dot pitch P_{DT}. As the dot pitch P_{DT} becomes smaller, an absorption band of the plasmon filter is more shifted to a short wavelength side, as the dot pitch P_{DT} becomes larger, an absorption band of the plasmon filter is more shifted to a long wavelength side.

Examples of a material for forming a dot may include a material for forming the above-described light reflecting layer, and specifically, aluminum (Al), silver (Ag), gold (Au), and copper (Cu). In addition, as a dielectric material, it is preferable to use a material having a low dielectric constant. Examples of the material may include a material having a low dielectric constant among the above-described materials for forming the insulating film, and specifically, SiO₂, a Low-K material, and the like. Further, examples of a material for forming the base layer may include the above-described materials for forming the insulating film.

Further, in a plasmon filter having a hole structure or a dot structure, a transmission band or an absorption band can be adjusted only by adjusting the pitch P_{HL} or P_{DT} of a hole or a dot in the second direction. Thus, for example, it is possible to individually set a transmission band or an absorption band for each light receiving element only by adjusting the pitch P_{HL} or P_{DT} of a hole or a dot in the second direction in a lithography step, and to increase the number of colors of a filter in fewer steps. In addition, the thickness of the plasmon filter is approximately 100 nm to 500 nm which is substantially the same as that of an organic material-based color filter, which leads to a good process affinity. A circular or elliptical shape can be exemplified as the planar shape of a hole or a dot. In addition, it is preferable that the long axis of an ellipse be parallel to the second direction.

Alternatively, the plasmon filter in the light receiving element and the like of the present disclosure is constituted by a plasmon resonance body in which a plurality of belt-like (lattice-like) conductive material layers are disposed lined up in the second direction as described above. The plasmon filter includes a conductive material layer and a base layer in order from an incident light side. The rectangular conductive material layer is formed on the base layer with a pitch P_{CL} in the second direction, and a transmission band of the plasmon filter changes depending on the pitch P_{CL}. In addition, the pitch of an adjacent rectangular conductive material layer in the second direction may be referred to as a “conductive material layer pitch P_{CL}” below. That is, as the conductive material layer pitch P_{CL} becomes smaller, a transmission band of the plasmon filter is more shifted to a short wavelength side, and the conductive material layer pitch P_{CL} becomes larger, a transmission band of the plasmon filter is more shifted to a long wavelength side.

Examples of a material for forming the conductive material layer may include a material for forming the above-described light reflecting layer, and specifically, aluminum (Al), silver (Ag), gold (Au), and copper (Cu). Further, examples of a material for forming the base layer may include the above-described materials for forming the insulating film. It is preferable that the base layer and the conductive material layer be covered with an interlayer film, and it is preferable to use a material having a low dielectric constant as a material for forming the interlayer film. Examples of the material may include a material having a low dielectric constant among the above-described materials for forming the insulating film, and specifically, SiO₂, a Low-K material, and the like.

In order to drive the light receiving element, for example, a plurality of various wirings (wiring layers) formed of aluminum (Al), copper (Cu), or the like are formed below the wire grid polarizing element. The wire grid polarizing element is connected to a semiconductor substrate through various wirings (wiring layers) and contact hole portions, and thus a predetermined potential can be applied to the wire grid polarizing element. Specifically, for example, the wire grid polarizing element is grounded. Examples of the semiconductor substrate may include a compound semiconductor substrate such as a silicon semiconductor substrate or an InGaAs substrate.

The light receiving element is formed on, for example, the silicon semiconductor substrate and the compound semiconductor substrate, or is formed above these conductor substrates. Alternatively, the photoelectric conversion portion constituting the light receiving element may be formed in a silicon layer (a crystalline silicon layer, an amorphous silicon layer, a microcrystalline silicon layer) formed on the silicon semiconductor substrate and the compound semiconductor substrate, a germanium layer, a selenium layer (a crystalline selenium layer, an amorphous selenium layer), an organic material layer, or a compound semiconductor layer (specifically, for example, an InP layer, an InAs layer, an InSb layer, an InGaAs layer, an InGaSb layer, an InGaP layer, an InGaAlP layer, an InGaAsP layer, an InAlP layer, an InAlAs layer, a GaAs layer, an AlGaAs layer), may be formed of a chalcopyrite-based compound (specifically, for example, CIGS (CuInGaSe), CIS (CuInSe₂), CuInS₂, CuAlS₂, CuAlSe₂, CuGaS₂, CuGaSe₂, AgAlS₂, AgAlSe₂, AgInS₂, AgInSe₂), and may be formed of a compound semiconductor such as CdSe, CdS, In₂Se₃, In₂S₃, Bi₂Se₃, Bi₂S₃, ZnSe, ZnS, PbSe, or PbS, and quantum dots formed of these materials can also be used for the photoelectric conversion portion.

In a case where an imaging element is constituted by a light receiving element, configurations and structures of a floating diffusion layer, an amplification transistor, a reset transistor, and a selection transistor that constitute a control unit controlling the driving of the imaging element can be the same as configurations and structures of a floating diffusion layer, an amplification transistor, a reset transistor, and a selection transistor in a control unit of the related art. A drive circuit can also have a known configuration and structure.

A waveguide structure may be provided between light receiving elements in a light receiving element group, or a light condensing pipe structure may be provided, and thus it is possible to achieve a reduction in optical crosstalk. Here, the waveguide structure is constituted by a thin film having a refractive index larger than a refractive index of a material for forming the interlayer insulating layer, the thin film being formed in a region (for example, a cylindrical region) positioned between photoelectric conversion portions of an interlayer insulating layer covering the photoelectric conversion portion. Light incident from above the photoelectric conversion portion is totally reflected by the thin film and reaches the photoelectric conversion portion. That is, an orthogonal projection image of the photoelectric conversion portion with respect to the substrate is positioned on the inner side of an orthogonal projection image of the thin film constituting the waveguide structure with respect to the substrate, and the orthogonal projection image of the photoelectric conversion portion with respect to the substrate is surrounded by the orthogonal projection image of the thin film constituting the waveguide structure with respect to the substrate. In addition, the light condensing pipe structure is constituted by a light shielding thin film formed of a metal material or an alloy material, the light shielding thin film being formed in a region (for example, a cylindrical region) positioned between photoelectric conversion portions of an interlayer insulating layer covering the photoelectric conversion portion. Light incident from above the photoelectric conversion portion is reflected by the thin film and reaches the photoelectric conversion portion. That is, an orthogonal projection image of the photoelectric conversion portion with respect to the substrate is positioned on the inner side of an orthogonal projection image of the thin film constituting the light condensing pipe structure with respect to the substrate, and the orthogonal projection image of the photoelectric conversion portion with respect to the substrate is surrounded by the orthogonal projection image of the thin film constituting the light condensing pipe structure with respect to the substrate.

In the light receiving device of the present disclosure, one light receiving element unit (one pixel) can be constituted by a plurality of light receiving elements (sub-pixels). For example, each sub-pixel includes one light receiving element. A configurations and structure of the light receiving element or the photoelectric conversion portion can be a well-known configuration and structure.

All of the light receiving elements constituting the light receiving device of the present disclosure may include a wire grid polarizing element, or some of the light receiving elements may include a wire grid polarizing element. It is possible to adopt a mode in which a light receiving element unit constituted by a plurality of light receiving elements has a Bayer array, and one light receiving element unit (one pixel) is constituted by four light receiving elements (four sub-pixels). However, an array of the light receiving element units is not limited to the Bayer array, and examples of the array may include other arrays such as an interline array, a G stripe RB checkered array, a G stripe RB complete checkered array, a checkered complementary color array, a stripe array, a diagonal stripe array, a primary color difference array, a field color difference sequential array, a frame color difference sequential array, a MOS type array, an improved MOS type array, a frame interleaved array, and a field interleaved array. The light receiving element may be constituted by a combination of a light receiving element for red light which is sensitive to red light, a light receiving element for green light which is sensitive to green light, and a light receiving element for blue light which is sensitive to blue light. Additionally, the light receiving element may be constituted by a combination of infrared light receiving elements which is sensitive to infrared light, may be configured as a solid-state imaging device that obtains an image of a single color in the light receiving device of the present disclosure, may be configured as a solid-state imaging device that obtains a combination of an image of a single color and an image based on infrared light, or may be configured as a solid-state imaging device that obtains an image based on infrared light.

In the light receiving element and the like of the present disclosure, it is possible to adopt a mode in which an on-chip microlens is disposed closer to a light incident side than to a wire grid polarizing element in each light receiving element. In this case, the on-chip microlens (OCL) can be constituted by a main on-chip microlens disposed above the wire grid polarizing element, and can also be constituted by a sub-on-chip microlens (inner lens, OPA) disposed above the wire grid polarizing element and a main on-chip microlens disposed above the sub-on-chip microlens (OPA).

In a case where the light receiving device of the present disclosure is used in a solid-state imaging device, examples of a light receiving element may include a CCD element, a CMOS image sensor, a contact image sensor (CIS), and a charge modulation device (CMD) type signal amplification type image sensor. The light receiving element is a surface irradiation type or rear surface irradiation type light receiving element. The solid-state imaging device can constitute, for example, a digital still camera, a video camera, a camcorder, a surveillance camera, a vehicle-mounted camera, a smart phone camera, a game user interface camera, a biometric camera, an endoscope, an angiography device by receiving infrared light, a skin measuring device that images skin, a microscope that images scalp, and a camera for monitoring the condition of fields and crops. It is possible to configure a solid-state imaging device that can acquire polarization information at the same time in addition to performing normal imaging. In addition, it is also possible to configure a solid-state imaging device that captures a three-dimensional image. In a case where a solid-state imaging device is constituted by the light receiving device of the present disclosure, a single panel color solid-state imaging device can be constituted by the solid-state imaging device.

### Example 1

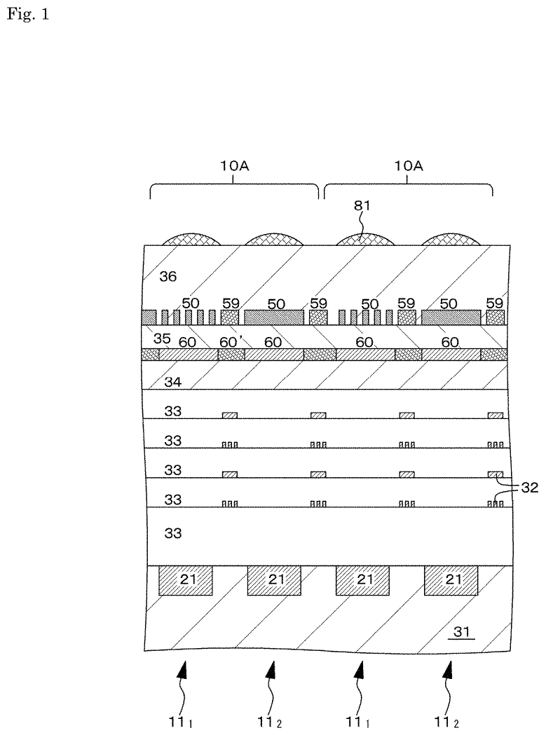

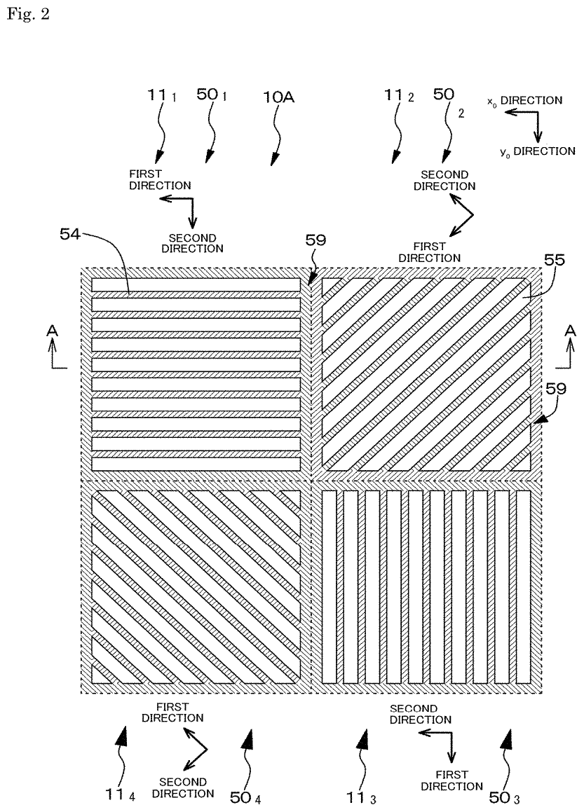

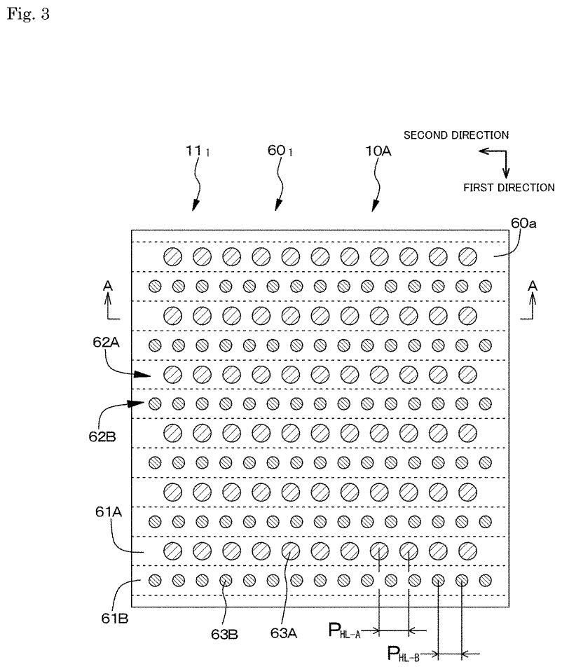

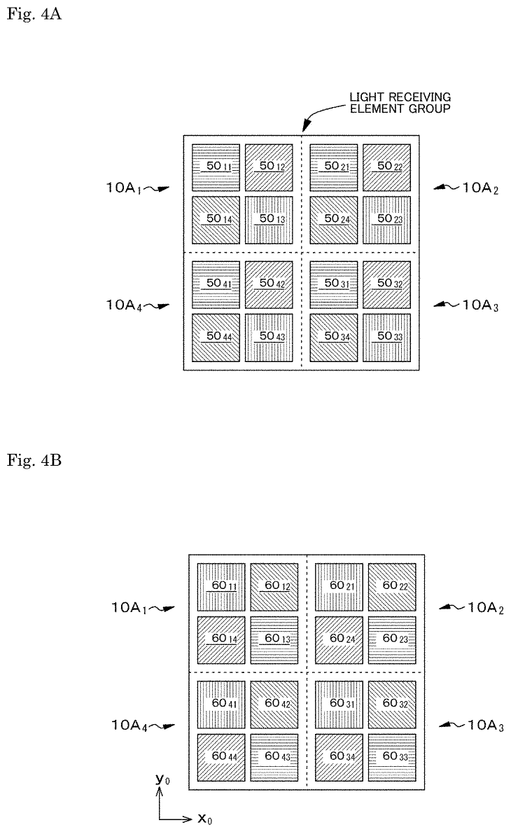

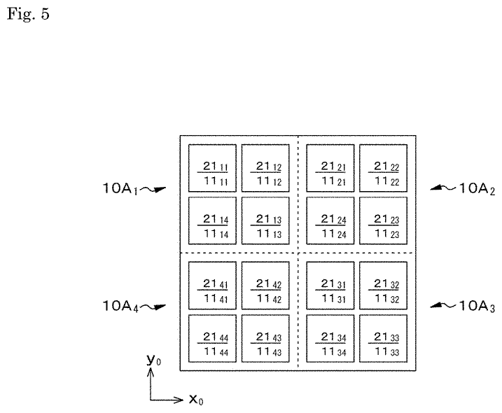

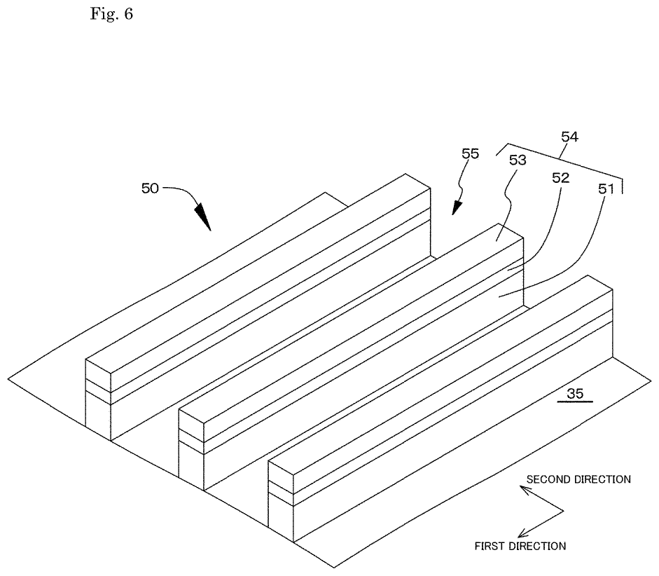

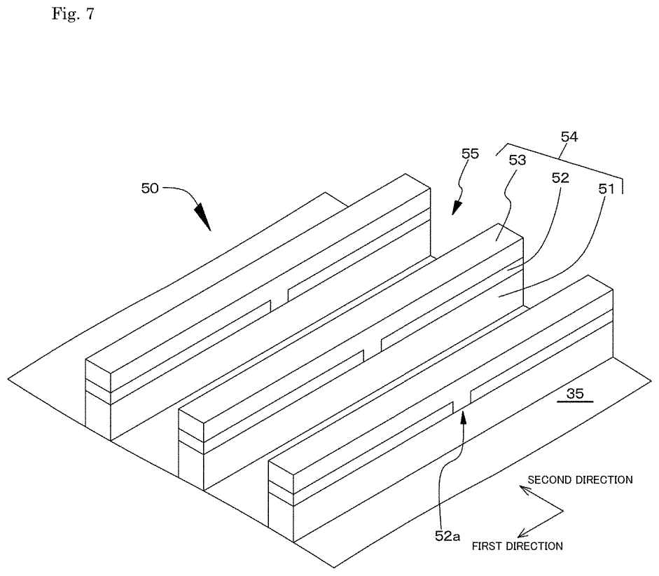

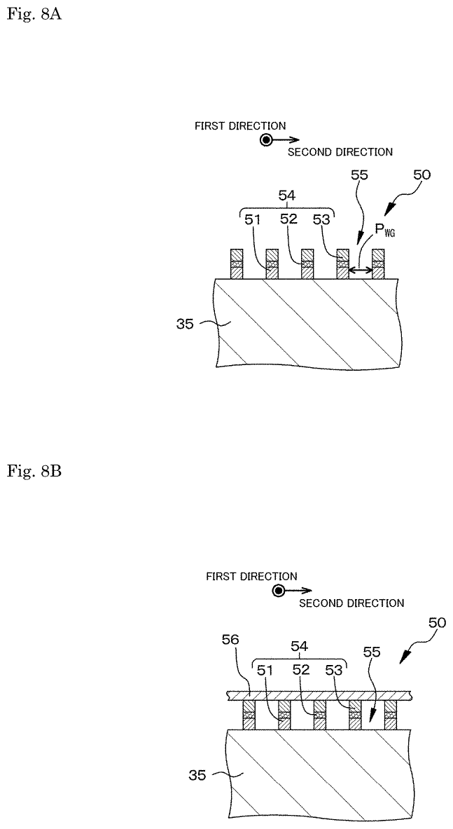

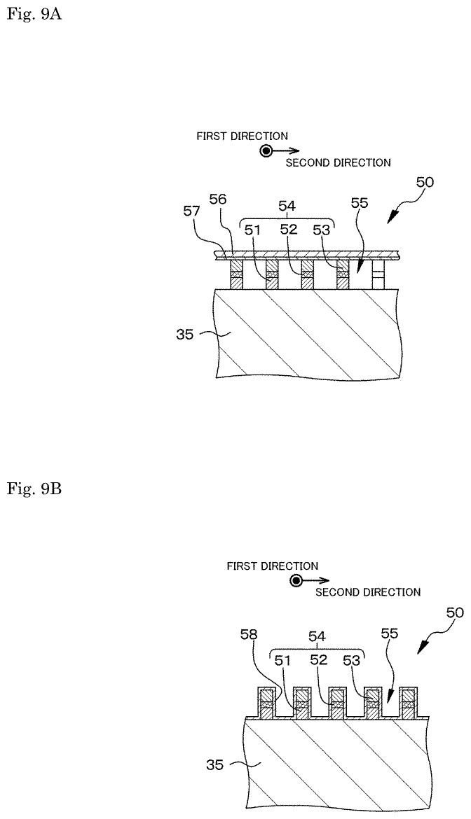

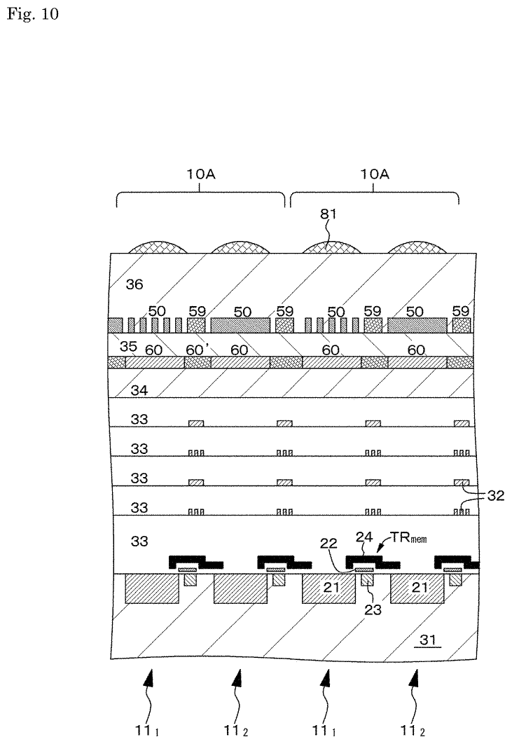

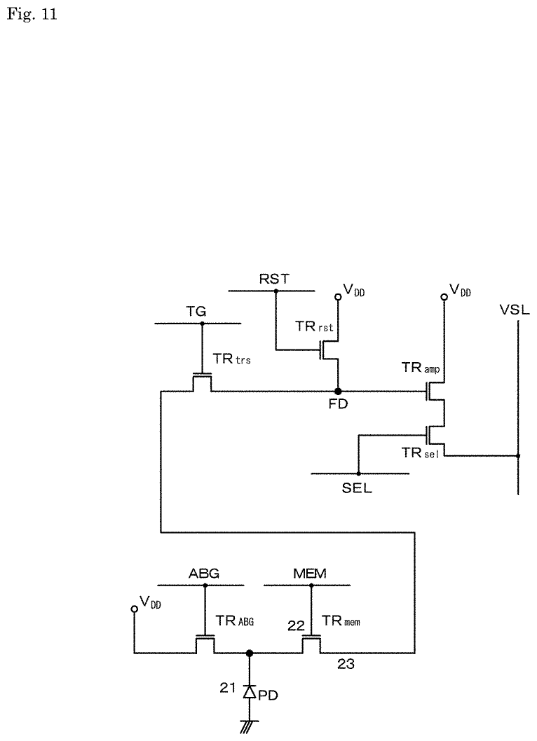

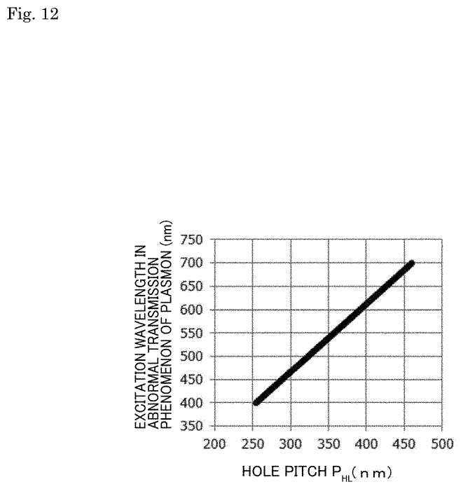

Example 1 relates to the light receiving device and the light receiving element of the present disclosure. A schematic partial cross-sectional view of the light receiving device in Example 1 which is taken along an arrow A-A in FIGS. 2 and 3 is illustrated in FIG. 1, a schematic plan view of wire grid polarizing elements included in four light receiving elements constituting a light receiving element unit in the light receiving device in Example 1 is illustrated in FIG. 2, and a schematic plan view of wavelength selection means constituting the light receiving element in Example 1 is illustrated in FIG. 3. In addition, a conceptual plan view illustrating the arrangement of the wire grid polarizing elements included in the four light receiving elements constituting the light receiving element unit in the light receiving device in Example 1 is illustrated in FIG. 4A, a conceptual plan view illustrating the arrangement of the wavelength selection means is illustrated in FIG. 4B, and a conceptual plan view illustrating the arrangement of photoelectric conversion portions is illustrated in FIG. 5. Further, a schematic perspective view of the wire grid polarizing element is illustrated in FIGS. 6 and 7, and a schematic partial cross-sectional view of the wire grid polarizing element is illustrated in FIGS. 8A, 8B, 9A, and 9B. Further, an equivalent circuit diagram of the photoelectric conversion portion in the light receiving device (solid-state imaging device) of Example 1 is illustrated in FIG. 11. Further, in a plasmon filter constituted by a plasmon resonance body constituted by a substrate in which a hole group is disposed in the second direction (the substrate is formed of aluminum), a graph in which a relationship between an excitation wavelength in an abnormal transmission phenomenon (surface plasmon resonance) of plasmon and a hole pitch P_{HL} of an adjacent hole in the second direction is obtained is illustrated in FIG. 12. Further, in FIGS. 4A and 4B and FIG. 5, one light receiving element unit is illustrated. Further, diagonal lines are added to holes to clearly indicate the holes in FIG. 3, and hatching lines are not added to a lower layer and interlayer insulating layer in FIG. 1 and FIG. 10 to be described later.

A light receiving element (photoelectric conversion element, imaging element) 11 in Example 1 is a rear surface irradiation type light receiving element, and includes a wire grid polarizing element 50, a wavelength selection means 60, and a photoelectric conversion portion 21 from a light incident side,

the wavelength selection means 60 is constituted by a plurality of wavelength selection members 61, and

the wavelength selection members 61 constituted by a plasmon filter transmit light having different wavelengths.

In addition, the light receiving device (imaging device, solid-state imaging device) in Example 1 is constituted by a light receiving element unit 10A constituted by the plurality of light receiving elements 11,

each of the light receiving elements 11 constituting each light receiving element unit 10A includes the wire grid polarizing element 50, the wavelength selection means 60, and the photoelectric conversion portion 21 from a light incident side, the wavelength selection means 60 is constituted by a plurality of wavelength selection members 61, and

the wavelength selection members 61 constituted by a plasmon filter transmit light having different wavelengths.

In each of the light receiving elements 11, an on-chip microlens 81 is disposed closer to the light incident side than to the wire grid polarizing element 50.

The light receiving device in Example 1 is constituted by a plurality of light receiving element groups arranged two-dimensionally as illustrated in FIGS. 4A and 4B and FIG. 5,

one light receiving element group is constituted by four light receiving element units 10A₁, 10A₂, 10A₃, and 10A₄ arranged in a 2×2 array,

the first light receiving element unit 10A₁ includes first wavelength selection means 60₁ (60₁₁, 60₁₂, 60₁₃, 60₁₄) that transmits light in a first wavelength range,

the second light receiving element unit 10A₂ includes second wavelength selection means 60₂ (60₂₁, 60₂₂, 60₂₃, 60₂₄) which transmits light in a second wavelength range,

the third light receiving element unit 10A₃ includes third wavelength selection means 60₃ (60₃₁, 60₃₂, 60₃₃, 60₃₄) which transmits light in a third wavelength range, and

the fourth light receiving element unit 10A₄ includes fourth wavelength selection means 60₄ (60₄₁, 60₄₂, 60₄₃, 60₄₄) which transmits light in a fourth wavelength range.

Specifically, one light receiving element group is constituted by, for example, four light receiving element units 10A₁, 10A₂, 10A₃, and 10A₄ disposed in a Bayer array, and examples of light to be received may include red light as light in a first wavelength range, green light as light in a second wavelength range and light in a third wavelength range, and blue light as light in a fourth wavelength range. Alternatively, examples thereof may include red light as light in a first wavelength range, green light as light in a second wavelength range, blue light as light in a third wavelength range, and infrared light as light in a fourth wavelength range.

Here, the first light receiving element unit 10A₁, the second light receiving element unit 10A₂, the third light receiving element unit 10A₃, and the fourth light receiving element unit 10A₄ are constituted by first light receiving elements 11₁₁, 11₂₁, 11₃₁, and 11₄₁, second light receiving elements 11₂₁, 11₂₂, 11₃₂, and 11₄₂, third light receiving elements 11₃₁, 11₂₃, 11₃₃, and 11₄₃, and fourth light receiving elements 11₄₁, 11₂₄, 11₃₄, and 11₄₄. That is, each light receiving element unit is constituted by four light receiving elements arranged in a 2×2 array. Then, a polarization direction of transmission performed by wire grid polarizing elements 50₁₁, 50₂₁, 50₃₁, and 50₄₁ constituting the first light receiving elements 11₁₁, 11₂₁, 11₃₁, and 11₄₁ of the first light receiving element unit 10A₁, the second light receiving element unit 10A₂, the third light receiving element unit 10A₃, and the fourth light receiving element unit 10A₄ is at α degrees,

a polarization direction of transmission performed by wire grid polarizing elements 50₁₂, 50₂₂, 50₃₂, and 50₄₂ constituting second light receiving elements 11₂₁, 11₂₂, 11₃₂, and 11₄₂ of the first light receiving element unit 10A₁, the second light receiving element unit 10A₂, the third light receiving element unit 10A₃, and the fourth light receiving element unit 10A₄ is at (α+45) degrees,

a polarization direction of transmission performed by wire grid polarizing elements 50₁₃, 50₂₃, 50₃₃, and 50₄₃ constituting third light receiving elements 11₃₁, 11₂₃, 11₃₃, and 11₄₃ of the first light receiving element unit 10A₁, the second light receiving element unit 10A₂, the third light receiving element unit 10A₃, and the fourth light receiving element unit 10A₄ is at (α+90) degrees, and

a polarization direction of transmission performed by wire grid polarizing elements 50₁₄, 50₂₄, 50₃₄, and 50₄₄ constituting fourth light receiving elements 11₄₁, 11₂₄, 11₃₄, and 11₄₄ of the first light receiving element unit 10A₁, the second light receiving element unit 10A₂, the third light receiving element unit 10A₃, and the fourth light receiving element unit 10A₄ is at (α+135) degrees.

Additionally,

the first light receiving elements 11₁₁, 11₂₁, 11₃₁, and 11₄₁ of the first light receiving element unit 10A₁, the second light receiving element unit 10A₂, the third light receiving element unit 10A₃, and the fourth light receiving element unit 10A₄ include wavelength selection means 60₁₁, 60₂₁, 60₃₁, and 60₄₁,

the second light receiving elements 11₂₁, 11₂₂, 11₃₂, and 11₄₂ of the first light receiving element unit 10A₁, the second light receiving element unit 10A₂, the third light receiving element unit 10A₃, and the fourth light receiving element unit 10A₄ include wavelength selection means 60₁₂, 60₂₂, 60₃₂, and 60₄₂,

the third light receiving elements 11₃₁, 11₂₃, 11₃₃, and 11₄₃ of the first light receiving element unit 10A₁, the second light receiving element unit 10A₂, the third light receiving element unit 10A₃, and the fourth light receiving element unit 10A₄ include wavelength selection means 60₁₃, 60₂₃, 60₃₃, and 60₄₃, and

the fourth light receiving elements 11₄₁, 11₂₄, 11₃₄, and 11₄₄ of the first light receiving element unit 10A₁, the second light receiving element unit 10A₂, the third light receiving element unit 10A₃, and the fourth light receiving element unit 10A₄ include wavelength selection means 60₁₄, 60₂₄, 60₃₄, and 60₄₄.

Further, in FIG. 4A, a hatching line indicates a first direction in the wire grid polarizing element, and in FIG. 4B, a hatching line indicates a second direction in the wavelength selection means. In addition, an angle formed between an angle α and the second direction can be substantially set to be any angle, but is set to 0 degrees in Example 1 or examples to be described below.

In the light receiving element 11 in Example 1, each wavelength selection member 61 is constituted by a periodic structure, and the periodic structure is constituted by a substrate 60a and a plurality of holes 63 provided in the substrate 60a. A polarization direction (second direction) of transmission performed by the wire grid polarizing element 50 and an arrangement direction of the plurality of holes 63 are parallel to each other. That is, the arrangement direction of the plurality of holes 63 is parallel to the second direction. In addition, a hole group 62 is constituted by the plurality of holes 63 arranged in the second direction and constituting each wavelength selection member 61, and a plurality of hole groups 62 are arranged in a first direction. Specifically, the hole groups 62 constituting each wavelength selection member 61 are alternately arranged in the first direction. Each wavelength selection member 61 is covered with an interlayer film 35, and the holes 63 are embedded with the interlayer film 35.

Further, in Example 1, it is assumed that the wavelength selection means 60 is constituted by two wavelength selection members 61, but is not limited thereto. The number of wavelength selection members can also be set to three or more. In addition, regarding one wavelength selection member, “A” is attached to the end of reference numeral, and regarding the other wavelength selection member, “B” is attached to the end of reference numeral. Further, regarding a third wavelength selection member, “C” is attached to the end of reference numeral. With such a configuration, the wavelength selection means 60 can transmit light having a plurality of (specifically, for example, two types) wavelengths.

In the light receiving device in Example 1, the photoelectric conversion portion 21 having a known configuration and structure is formed in the silicon semiconductor substrate 31 by a known method. Light receiving elements 11₁₁, 11₁₂, 11₁₃, and 11₁₄ constituting the first light receiving element unit 10A₁ include photoelectric conversion portions 21₁₁, 21₁₂, 21₁₃, and 21₁₄. Light receiving elements 11₂₁, 11₂₂, 11₂₃, and 11₂₄ constituting the second light receiving element unit 10A₂ include photoelectric conversion portions 21₂₁, 21₂₂, 21₂₃, and 21₂₄. Light receiving elements 11₃₁, 11₃₂, 11₃₃, and 11₃₄ constituting the third light receiving element unit 10A₃ include photoelectric conversion portions 21₃₁, 21₃₂, 21₃₃, and 21₃₄. Light receiving elements 11₄₁, 11₄₂, 11₄₃, and 11₄₄ constituting the fourth light receiving element unit 10A₄ include photoelectric conversion portions 21₄₁, 21₄₂, 21₄₃, and 21₄₄.

The photoelectric conversion portion 21 is covered with an interlayer insulating layer (lower layer and interlayer insulating layer 33), a base insulating layer 34 is formed on the lower layer and interlayer insulating layer 33, and a wavelength selection means 60 and a wavelength selection means and extended portion 60′ are formed on the base insulating layer 34. The wavelength selection means and extended portion 60′ is provided in a region between the light receiving elements 11, and has the same configuration and structure as those of the wavelength selection means 60 except that holes are not formed therein. The wavelength selection means 60 and the wavelength selection means and extended portion 60′ including the holes 63 are covered with the interlayer film 35, and the wire grid polarizing element 50 is formed on the interlayer film 35. An upper layer and interlayer insulating layer 36 is formed on the wire grid polarizing element 50, and an on-chip microlens 81 is disposed on the upper layer and interlayer insulating layer 36. In addition, five lower layer and interlayer insulating layers 33 and four wiring layers 32 are illustrated in the drawing. However, the present disclosure is not limited thereto, and the numbers of lower layer and interlayer insulating layers 33 and wiring layers 32 are arbitrary.

As illustrated in FIG. 8A, the wire grid polarizing element 50 has a line-and-space structure. A line portion 54 of the wire grid polarizing element 50 is constituted by a laminated structure (first laminated structure) in which a light reflecting layer 51 formed of a first conductive material (specifically, aluminum (Al)), an insulating film 52 formed of SiO₂, and a light absorbing layer 53 formed of a second conductive material (specifically, tungsten (W)) are laminated from a side (a photoelectric conversion portion side in Example 1) which is opposite to a light incident side. The insulating film 52 is formed on the entire top surface of the light reflecting layer 51, and the light absorbing layer 53 is formed on the entire top surface of the insulating film 52. Specifically, the light reflecting layer 51 is formed of aluminum (Al) having a thickness of 150 nm, the insulating film 52 is formed of SiO₂ having a thickness of 25 nm or 50 nm, and the light absorbing layer 53 is formed of tungsten (W) having a thickness of 25 nm. The light reflecting layer 51 has a function as a polarizer, attenuates a polarized wave having an electric field component in a direction parallel to an extension direction (first direction) of the light reflecting layer 51, and transmits a polarized wave having an electric field component in a direction (second direction) orthogonal to the extension direction of the light reflecting layer 51 among light incident on the wire grid polarizing element 50. The first direction is a light absorption axis of the wire grid polarizing element 50, and the second direction is a light transmission axis of the wire grid polarizing element 50. A base film formed of Ti, TiN, or a laminated structure of Ti/TiN is formed between the base insulating layer 34 and the light reflecting layer 51, but the base film is not illustrated in the drawing.

The light reflecting layer 51, the insulating film 52, and the light absorbing layer 53 are common to the light receiving elements 11. A frame portion 59 is constituted by a laminated structure (second laminated structure) including the light reflecting layer 51, the insulating film 52, and the light absorbing layer 53 except that a space portion 55 is not provided. That is, as illustrated in the schematic plan view of FIG. 2, the frame portion 59 surrounding the wire grid polarizing element 50 is provided, and the frame portion 59 and the line portion 54 of the wire grid polarizing element 50 are connected to each other. In this manner, the frame portion 59 has the same structure as that of the line portion 54 of the wire grid polarizing element 50 and also functions as a light shielding portion.

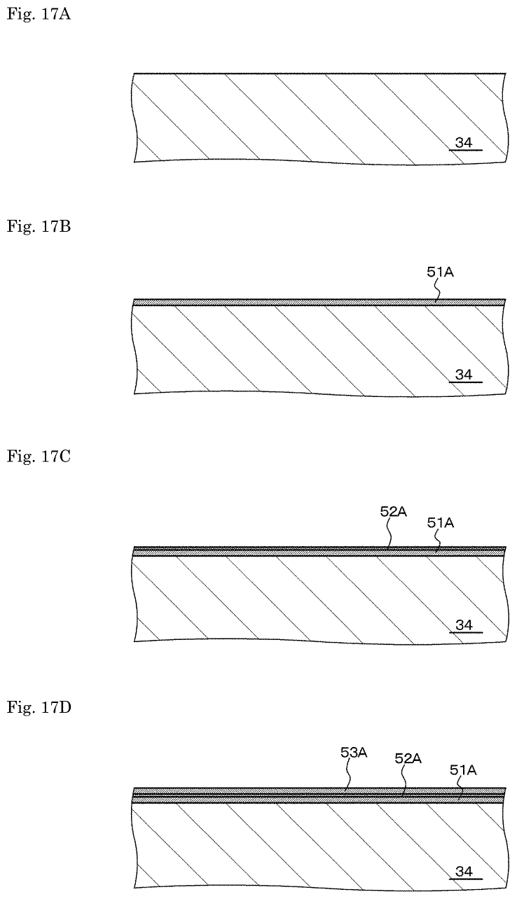

The wire grid polarizing element 50 can be manufactured by the following method. That is, a base film (not illustrated) formed of Ti, TiN, or a laminated structure of Ti/TiN and a light reflecting layer formation layer 51A formed of a first conductive material (specifically, aluminum) are provided on the base insulating layer 34 on the basis of a vacuum vapor deposition method (see FIGS. 17A and 17B). Next, an insulating film formation layer 52A is provided on the light reflecting layer formation layer 51A, and a light absorbing layer formation layer 53A formed of a second conductive material is provided on the insulating film formation layer 52A. Specifically, the insulating film formation layer 52A formed of SiO₂ is formed on the light reflecting layer formation layer 51A on the basis of a CVD method (see FIG. 17C). The light absorbing layer formation layer 53A formed of tungsten (W) is formed on the insulating film formation layer 52A by a sputtering method. In this manner, a structure illustrated in FIG. 17D can be obtained.

Thereafter, by patterning the light absorbing layer formation layer 53A, the insulating film formation layer 52A, the light reflecting layer formation layer 51A, and the base film on the basis of lithography technology and dry etching technology, it is possible to obtain the wire grid polarizing element 50 having a line-and-space structure in which a plurality of line portions (laminated structure) 54 of the belt-like light reflecting layer 51, the insulating film 52, and the light absorbing layer 53 are separately arranged in parallel. Thereafter, the interlayer film 35 may be formed to cover the wire grid polarizing element 50 on the basis of a CVD method. The wire grid polarizing element 50 is surrounded by the frame portion 59 (see FIG. 2) constituted by the light reflecting layer 51, the insulating film 52, and the light absorbing layer 53.

As a modification example of the wire grid polarizing element 50, it is possible to adopt a configuration in which a protection film 56 formed on the wire grid polarizing element 50 is provided as illustrated in a schematic partial cross-sectional view of FIG. 8B, and the space portion 55 of the wire grid polarizing element 50 is a void. That is, a portion or the entirety of the space portion 55 is filled with air. In Example 1, specifically, the entire space portion 55 is filled with air.

In addition, as illustrated in a schematic partial cross-sectional view of FIG. 9A, it is also possible to adopt a configuration in which a second protection film 57 is formed between the wire grid polarizing element 50 and the protection film 56. When a refractive index of a material for forming the protection film 56 is set to be n₁′, ad a refractive index of a material for forming the second protection film 57 is set to be n₂′, a relationship of n₁′>n₂′ is satisfied. Here, for example, the protection film 56 is formed of SiN (n₁′=2.0), and the second protection film 57 is formed of SiO₂ (n₂′=1.5). In the drawing, a bottom face (a surface facing the base insulating layer 34) of the second protection film 57 is illustrated in a flat state, but the bottom face of the second protection film 57 may have a convex shape toward the space portion 55, or the bottom face of the second protection film 57 may have a concave shape toward the protection film 56 or may be recessed in a wedge shape.

In such a structure, the wire grid polarizing element 50 having a line-and-space structure is obtained, and then the second protection film 57 formed of SiO₂ and having an average thickness 0.01 μm to 10 μm is formed on the entire surface on the basis of a CVD method. An upper portion of the space portion 55 positioned between the line portion 54 and the line portion 54 is closed by the second protection film 57. Next, the protection film 56 formed of SiN and having an average thickness of 0.1 μm to 10 μm is formed on the second protection film 57 on the basis of a CVD method. It is possible to obtain a photoelectric conversion portion having high reliability by the protection film 56 formed of SiN. However, SiN has a relatively high relative dielectric constant, and thus a reduction in an average refractive index nₐᵥₑ is achieved by forming the second protection film 57 formed of SiO₂.

It is possible to reduce the value of the average refractive index nₐᵥₑ by forming the space portion of the wire grid polarizing element as a void in this manner (specifically, the space portion is filled with air), and consequently, it is possible to achieve an improvement in transmittance and an improvement in an extinction ratio in the wire grid polarizing element. In addition, the value of the formation pitch P_{WG} can be increased, and thus it is possible to achieve an improvement in the manufacturing yield of the wire grid polarizing element. Furthermore, when a protection film is formed on the wire grid polarizing element, it is possible to provide a photoelectric conversion portion and a light receiving device with high reliability. In addition, by connecting the frame portion and the line portion of the wire grid polarizing element and by forming the frame portion to have the same structure as that of the line portion of the wire grid polarizing element, it is possible to form a stable, homogeneous and uniform wire grid polarizing element. Thus, it is possible to solve a problem that peeling occurs in the outer peripheral portion of the wire grid polarizing element corresponding to four corners of the photoelectric conversion portion, a problem that there is a difference between the structure of the outer peripheral portion of the wire grid polarizing element and the structure of the central portion of the wire grid polarizing element, which results in a deterioration in the performance of the wire grid polarizing element itself, and a problem that light incident on the outer peripheral portion of the wire grid polarizing element easily leaks to an adjacent photoelectric conversion portion having a different polarization direction, and it is possible to provide a photoelectric conversion portion and a light receiving device with high reliability.

The wire grid polarizing element can be configured to have a structure in which an insulating film is omitted, that is, a configuration in which a light reflecting layer (formed of, for example, aluminum) and a light absorbing layer (formed of, for example, tungsten) are laminated from a side opposite to a light incident side. Alternatively, the wire grid polarizing element can also be constituted by a single conductive light shielding material layer. Examples of a material for forming the conductive light shielding material layer may include a conductive material having a complex refractive index in a wavelength band in which the photoelectric conversion portion has sensitivity, such as aluminum (Al), copper (Cu), gold (Au), silver (Ag), platinum (Pt), tungsten (W), or alloys containing these metals.

In some cases, as illustrated in a schematic partial cross-sectional view of the wire grid polarizing element in FIG. 9B, a third protection film 58 formed of, for example, SiO₂ may be formed on a side surface of the line portion 54 facing the space portion 55. That is, the space portion 55 is filled with air, and the third protection film 58 is provided in the space portion. The third protection film 58 is formed on the basis of, for example, an HDP-CVD method, and thus a thinner third protection film 58 can be conformally formed on the side surface of the line portion 54.

In some cases, as illustrated in a schematic perspective view according to a modification example of the wire grid polarizing element in FIG. 7, it is also possible to adopt a configuration in which a portion of the insulating film 52 is notched, and the light reflecting layer 51 and the light absorbing layer 53 are in contact with each other at a notch portion 52a of the insulating film 52.

In the semiconductor substrate 31, a memory portion TRₘₑₘ which is connected to the photoelectric conversion portion 21 and temporarily stores charge generated in the photoelectric conversion portion 21 may be formed. A schematic partial cross-sectional view according to a modification example of the light receiving element in Example 1 is illustrated in FIG. 10, and the memory portion TRₘₑₘ is constituted by a photoelectric conversion portion 21, a gate portion 22, a channel formation region, and a high-concentration impurity region 23. The gate portion 22 is connected to a memory selection line MEM. In addition, the high-concentration impurity region 23 is formed in the silicon semiconductor substrate 31 so as to be separated from the photoelectric conversion portion 21 by a well-known method. The light shielding film 24 is formed above the high-concentration impurity region 23. That is, the high-concentration impurity region 23 is covered with the light shielding film 24. Thus, light is prevented from being incident on the high-concentration impurity region 23. It is possible to easily realize a so-called global shutter function by including the memory portion TRₘₑₘ that temporarily stores charge. Examples of a material for forming the light shielding film 24 may include chromium (Cr), copper (Cu), aluminum (Al), tungsten (W), and a resin that does not transmit light (for example, a polyimide resin).

A transfer transistor TRₜᵣₛ illustrated in only FIG. 11 is constituted by a gate portion connected to a transfer gate line TG, a channel formation region, one source/drain region connected to the high-concentration impurity region 23 (or sharing a region with the high-concentration impurity region 23), and the other source/drain region constituting a floating diffusion layer FD.

A reset transistor TRᵣₛₜ illustrated in only FIG. 11 is constituted by a gate portion, a channel formation region, and a source/drain region. The gate portion of the reset transistor TRᵣₛₜ is connected to a reset line RST, one source/drain region of the reset transistor TRᵣₛₜ is connected to a power supply V_{DD}, and the other source/drain region also serves as a floating diffusion layer FD.

An amplification transistor TRₐₘₚ illustrated in only FIG. 11 is constituted by a gate portion, a channel formation region, and a source/drain region. The gate portion is connected to the other source/drain region (floating diffusion layer FD) of the reset transistor TRᵣₛₜ through a wiring layer. In addition, one source/drain region is connected to the power supply V_{DD}.

A selection transistor TRₛₑₗ illustrated in only FIG. 11 is constituted by a gate portion, a channel formation region, and a source/drain region. The gate portion is connected to a selection line SEL. In addition, one source/drain region shares a region with the other source/drain region constituting the amplification transistor TRₐₘₚ, and the other source/drain region is connected to a signal line (data output line) VSL (117).

The photoelectric conversion portion 21 is connected to one source/drain region of a charge emission control transistor TR_{ABG}. The gate portion of the charge emission control transistor TR_{ABG} is connected to a charge emission control transistor control line ABG, and the other source/drain region is connected to the power supply V_{DD}.

A series of operations including charge storage, a reset operation, and charge transfer of the photoelectric conversion portion 21 is the same as a series of operations including charge storage, a reset operation, and charge transfer in a photoelectric conversion portion of the related art, and thus detailed description thereof will be omitted.

The photoelectric conversion portion 21, the memory portion TRₘₑₘ, the transfer transistor TRₜᵣₛ, the reset transistor TRᵣₛₜ, the amplification transistor TRₐₘₚ, the selection transistor TRₛₑₗ, and the charge emission control transistor TR_{ABG} are covered with the lower layer and interlayer insulating layer 33.

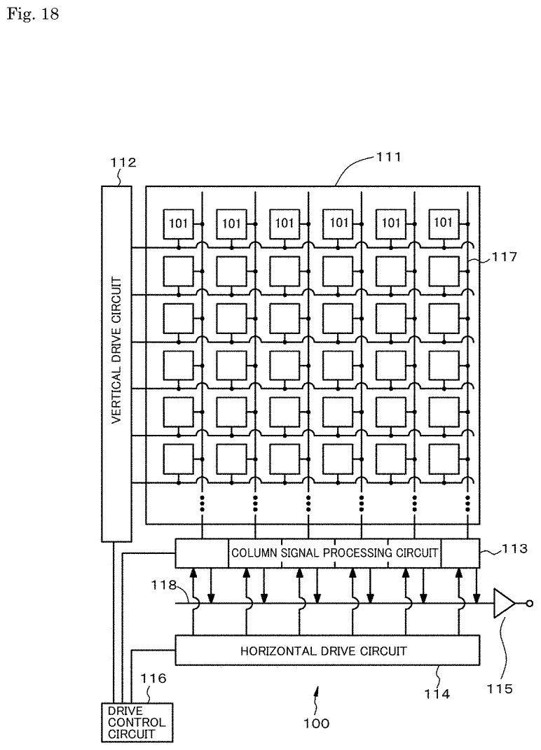

FIG. 18 illustrates a conceptual diagram of a solid-state imaging device in a case where the light receiving device in Example 2 is applied to the solid-state imaging device. A solid-state imaging device 100 in Example 1 includes an imaging region (effective pixel region) 111 in which photoelectric conversion portions 101 are arranged in a two-dimensional array, a vertical drive circuit 112 disposed in a peripheral region and serving as a drive circuit (peripheral circuit), a column signal processing circuit 113, a horizontal drive circuit 114, an output circuit 115, a drive control circuit 116, and the like. It is needless to say that these circuits can be constituted by well-known circuits and can be configured using other circuit configurations (for example, various circuits used in a CCD imaging device and a CMOS imaging device in the related art). In FIG. 18, reference numeral “101” in the photoelectric conversion portion 101 is displayed for only one row.

The drive control circuit 116 generates a clock signal or a control signal that serves as a reference for the operation of the vertical drive circuit 112, the column signal processing circuit 113, and the horizontal drive circuit 114 on the basis of a vertical synchronization signal, a horizontal synchronization signal, and a master clock. The generated clock signal or control signal is input to the vertical drive circuit 112, the column signal processing circuit 113, and the horizontal drive circuit 114.

The vertical drive circuit 112, which is constituted by, for example, a shift register, sequentially selectively scans the photoelectric conversion portions 101 in the imaging region 111 in units of rows in the vertical direction. A pixel signal (image signal) based on a current (signal) generated in accordance with the amount of received light in the photoelectric conversion portions 101 is transmitted to the column signal processing circuit 113 through a signal line (data output line) 117 and VSL.

The column signal processing circuit 113 is disposed, for example, for each column of the photoelectric conversion portions 101, and signal processing such as noise removal and signal amplification is performed on image signals output from the photoelectric conversion portions 101 of one row using signals output from black reference pixels (not illustrated in the drawing, and formed around effective pixel region) for each photoelectric conversion portion. A horizontal selection switch (not illustrated) is connected between an output end of the column signal processing circuit 113 and the horizontal signal line 118.

The horizontal drive circuit 114, which is constituted by, for example, a shift register, sequentially outputs horizontal scanning pulses and thus sequentially selects each of the column signal processing circuits 113, and outputs signals from each of the column signal processing circuits 113 to the horizontal signal line 118.

The output circuit 115 performs signal processing on signals that are sequentially supplied through the horizontal signal line 118 from each of the column signal processing circuits 113, and outputs the processed signals

In the light receiving element constituting the above-described light receiving device in Example 1 or in the light receiving element in Example 1, the wire grid polarizing element, the wavelength selection means, and the photoelectric conversion portions are provided from a light incident side, the wavelength selection means is constituted by a plurality of wavelength selection members, and each of the wavelength selection members constituted by a plasmon filter transmits light having different wavelengths, and thus one light receiving element (imaging element) can receive light having a plurality of wavelengths or can receive light in a wide wavelength band. In addition, when an image is obtained by the light receiving device (imaging device) of the present disclosure by irradiating an object or the like with light (visible light, and infrared light including near-infrared light, and far-infrared light) emitted from a predetermined light source used for measurement, evaluation, and analysis, an image can be reliably obtained even under an environment where the influence of outside light (for example, sunlight or light of an indoor lamp) is not negligible. Furthermore, as will be described below, light having a desired wavelength can be received with an extremely high efficiency.

In the light receiving device in Example 1, one light receiving element group includes four light receiving element units 10A₁, 10A₂, 10A₃, and 10A₄ disposed in a 2×2 array, and one light receiving element unit 10A includes four light receiving elements 11. Thus, when the amount of light of “100” is incident on one light receiving element unit 10A, the amount of light incident on the light receiving elements 11 is “25”. Since the amount of light passing through the wire grid polarizing element 50 is empirically 40%, the amount of light passing through the wire grid polarizing element 50 is 25×0.4=10.

Since an arrangement direction of the plurality of holes 63 is parallel to a second direction (polarization direction), that is, since a polarization direction of incident light on the wavelength selection member 61 is aligned, the absorption of light in the wavelength selection member 61 is negligible, and the amount of light having passed through the wavelength selection member 61 is 25×0.4×1=10.

On the other hand, in a case where a color filter layer is disposed instead of the wavelength selection member 61, the amount of light passing through the color filter layer is empirically 70%, and thus 25×0.4×0.7=7.

Further, in a case where a light receiving element in which the wavelength selection means 60, the wire grid polarizing element 50, and the photoelectric conversion portion 21 are disposed from a light incident side is assumed, a polarization direction of incident light on the wavelength selection means 60 is not aligned, the amount of light passing through the wavelength selection means 60 is empirically 40%, and the amount of light passing through the wire grid polarizing element 50 is 25×0.4×0.4=4.

From the above-described consideration, it can be understood that incident light can be taken into the photoelectric conversion portion with high efficiency by disposing the wire grid polarizing element 50, the wavelength selection means 60, and the photoelectric conversion portion 21 in this order from a light incident side.

Further, in the light receiving device in Example 1, one light receiving element unit includes four types of wire grid polarizing elements, and thus it is possible to configure a light receiving device (solid-state imaging device) capable of acquiring polarization information. That is, a polarization separation function for spatially performing polarization separation of polarization information of incident light can be given to the light receiving device (solid-state imaging device). Specifically, a light intensity, a polarization component intensity, and a polarization direction can be obtained in each light receiving element (imaging element). For example, by applying desired processing to a portion of an image obtained by imaging the sky or a windowpane, a portion of an image obtained by imaging the water surface, or the like, it is possible to emphasize or reduce a polarization component or to separate various polarization components, and it is possible to improve the contrast of an image and delete unnecessary information.

### Example 2

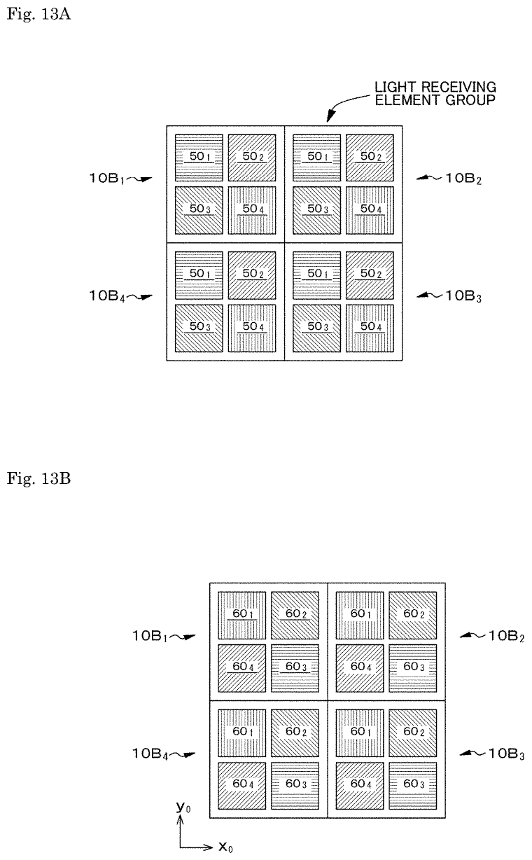

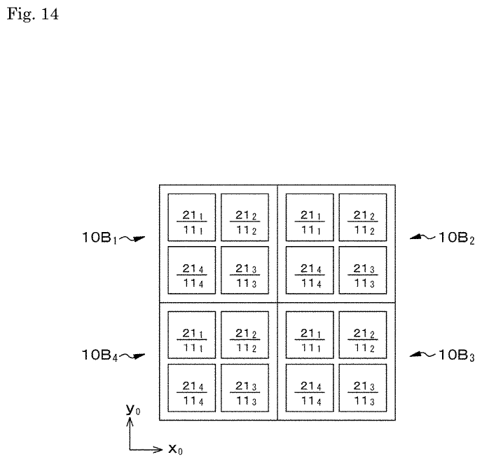

Example 2 is a modification of Example 1. A conceptual plan view illustrating the arrangement of wire grid polarizing elements included in four light receiving elements constituting a light receiving element group in a light receiving device in Example 2 is illustrated in FIG. 13A, a conceptual plan view illustrating the arrangement of wavelength selection means is illustrated in FIG. 13B, and a conceptual plan view illustrating the arrangement of photoelectric conversion portions is illustrated in FIG. 14. In these drawings, light receiving element groups 10B disposed in a 2×2 array are illustrated.

Unlike Example 1 the light receiving device in Example 2 is constituted by the plurality of light receiving element groups 10B disposed two-dimensionally. Each light receiving element group 10B includes four light receiving elements (a first light receiving element 11₁, a second light receiving element 11₂, a third light receiving element 11₃, and a fourth light receiving element 11₄) which are disposed in a 2×2 array. A polarization direction of transmission performed by a wire grid polarizing element 50₁ constituting the first light receiving element 11₁ is at α degrees, a polarization direction of transmission performed by a wire grid polarizing element 50₂ constituting the second light receiving element 11₂ is at (α+45) degrees, a polarization direction of transmission performed by a wire grid polarizing element 50₃ constituting the third light receiving element 11₃ is at (α+90) degrees, and a polarization direction of transmission performed by a wire grid polarizing element 50₄ constituting the fourth light receiving element 11₄ is at (α+135) degrees. In addition, the first light receiving element 11₁ includes wavelength selection means 60₁ and a photoelectric conversion portion 21₁, the second light receiving element 11₂ includes wavelength selection means 60₂ and a photoelectric conversion portion 21₂, the third light receiving element 11₃ includes wavelength selection means 60₃ and a photoelectric conversion portion 21₃, and the fourth light receiving element 11₄ includes wavelength selection means 60₄ and a photoelectric conversion portion 21₄. With such a configuration, the wavelength selection means 60 can transmit light having a plurality of types (specifically, two types) of wavelengths, and the wavelength selection means 60 can transmit light having a desired wavelength band.

Except for the above-described points, the configurations and structures of the light receiving element and the light receiving device in Example 2 can be the same as the configurations and structures of the light receiving element and the light receiving device in Example 1, and thus detailed description thereof will be omitted.

### Example 3

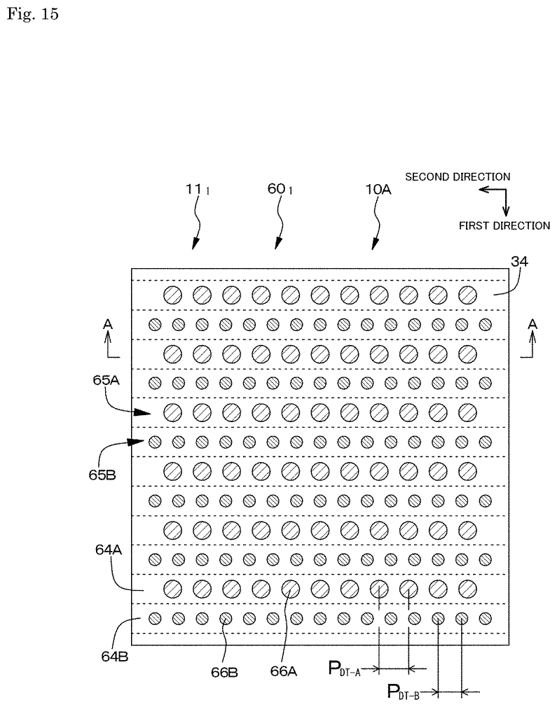

Example 3 is a modification of Example 1 and Example 2. A schematic plan view of wavelength selection means included in a light receiving element constituting a light receiving element unit in a light receiving device in Example 3 is illustrated in FIG. 15.

In the light receiving element in Example 3,

each wavelength selection member 64 is constituted by a periodic structure, and the periodic structure includes a base layer (specifically, a base insulating layer 34 in Example 3), dots 66 provided on the base layer (base insulating layer 34), and a dielectric material (specifically, an interlayer film 35 in Example 3) provided on the base layer (base insulating layer 34) and filled between the dots 66. A polarization direction (second direction) of transmission performed by the wire grid polarizing element 50 and an arrangement direction of the plurality of dots 66 are parallel to each other. That is, the arrangement direction of the plurality of dots 66 is parallel to a second direction. Further, a dot group 65 is constituted by the plurality of dots 66 arranged in the second direction and constituting each wavelength selection member 64, the plurality of dot groups 65 are arranged in a first direction, and the dot groups 65 constituting each wavelength selection member 64 are alternately arranged in the first direction. Further, in the example illustrated in FIG. 15, it is assumed that two types of dot groups 65 are arranged in the first direction.

In this manner, in the light receiving element in Example 3, a plasmon filter is constituted by a plasmon resonance body in which the dot groups 65 are disposed in the second direction. The plasmon filter functions as a filter that absorbs light in a predetermined wavelength band. An absorption band of light absorbed by the plasmon filter depends on a dot pitch P_{DT}. In addition, the diameter of the dot 66 is adjusted in accordance with the dot pitch P_{DT}. As the dot pitch P_{DT} becomes smaller, an absorption band of the plasmon filter is more shifted to a short wavelength side, and as the dot pitch P_{DT} becomes larger, the absorption band of the plasmon filter is more shifted to a long wavelength side.

Examples of a material for forming the dot may include aluminum (Al) and copper (Cu). In addition, specific examples of a material for forming the interlayer film may include SiO₂ and a Low-K material.

With such a configuration, the wavelength selection means 60 can transmit light having a plurality of types (specifically, two types) of wavelengths, and the wavelength selection means 60 can transmit light having a desired wavelength band. In addition, the order of a wavelength selection member 64A, a wavelength selection member 64B, and a wavelength selection member 64C arranged in the first direction is substantially arbitrary.

Except for the above-described points, the configurations and structures of the light receiving element and the light receiving device in Example 3 can be the same as the configurations and structures of the light receiving elements and the light receiving devices in Example 1 and Example 2, and thus detailed description thereof will be omitted.

### Example 4

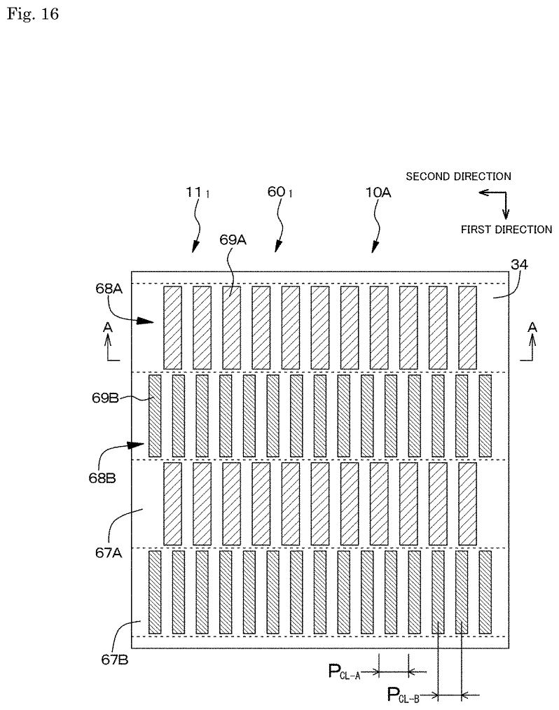

Example 4 is also a modification of Example 1 and Example 2. A schematic plan view of wavelength selection means included in a light receiving element constituting a light receiving element unit in a light receiving device in Example 4 is illustrated in FIG. 16.

In the light receiving element in Example 4,

each wavelength selection member 67 is constituted by a periodic structure, and the periodic structure includes a base layer (specifically, a base insulating layer 34 in Example 4) and a plurality of belt-like (lattice-like) conductive material layers 69 provided on the base layer (base insulating layer 34). A polarization direction (second direction) of transmission performed by the wire grid polarizing element 50 and a repetition direction of the belt-like conductive material layers 69 are parallel to each other. In other words, an extension direction of the belt-like conductive material layers 69 is a first direction, and the repetition direction of the belt-like conductive material layers 69 is the second direction. Further, a conductive material layer group 68 is constituted by the plurality of belt-like conductive material layers 69 arranged in the second direction and constituting each wavelength selection member 67, the plurality of conductive material layer groups 69 are arranged in the first direction, and the conductive material layer groups 68 constituting each wavelength selection member 67 are alternately arranged in the first direction. Further, in the example illustrated in FIG. 16, two types of conductive material layer groups 68 are arranged in the first direction.

In this manner, in the light receiving element in Example 4, a plasmon filter is constituted by a plasmon resonance body in which the plurality of belt-like (lattice-like) conductive material layers 69 are disposed lined up in the second direction. In the plasmon filter, a rectangular conductive material layer 69 formed of, for example, aluminum (Al) is formed on a base layer (base insulating layer 34) with a conductive material layer pitch P_{CL} in the second direction, and a transmission band of the plasmon filter changes depending on the conductive material layer pitch P_{CL}. That is, as the conductive material layer pitch P_{CL} becomes smaller, a transmission band of the plasmon filter is more shifted to a short wavelength side, and as the conductive material layer pitch P_{CL} becomes larger, a transmission band of the plasmon filter is more shifted to a long wavelength side.

With such a configuration, the wavelength selection means 60 can transmit light having a plurality of types (specifically, two types) of wavelengths, and the wavelength selection means 60 can transmit light having a desired wavelength band.

Except for the above-described points, the configurations and structures of the light receiving element and the light receiving device in Example 4 can be the same as the configurations and structures of the light receiving elements and the light receiving devices in Example 1 and Example 2, and thus detailed description thereof will be omitted.

### Example 5

The light receiving device (imaging device) of the present disclosure can be applied to various products. For example, the light receiving device (imaging device) of the present disclosure may be realized as a device mounted on any type of moving body such as an automobile, an electric vehicle, a hybrid electric vehicle, a motorcycle, a bicycle, a personal mobility, an airplane, a drone, a ship, and a robot.

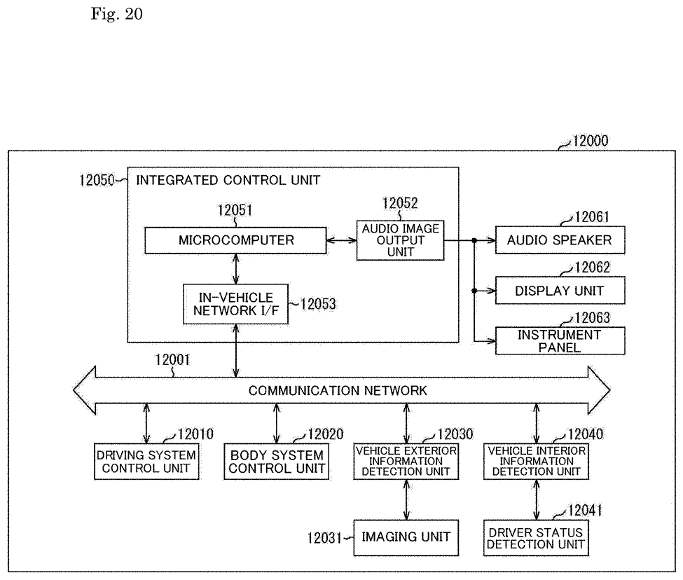

FIG. 20 is a block diagram illustrating a schematic configuration example of a vehicle control system which is an example of a moving body control system to which the technology according to the present disclosure can be applied.

A vehicle control system 12000 includes a plurality of electronic control units connected to each other through a communication network 12001. In the example illustrated in FIG. 20, the vehicle control system 12000 includes a drive system control unit 12010, a body system control unit 12020, a vehicle exterior information detection unit 12030, a vehicle interior information detection unit 12040, and an integrated control unit 12050. In addition, as a functional configuration of the integrated control unit 12050, a microcomputer 12051, an audio image output unit 12052, and an in-vehicle network interface (I/F) 12053 are illustrated.

The drive system control unit 12010 controls operations of devices related to a drive system of a vehicle according to various programs. For example, the drive system control unit 12010 functions as a driving force generation device for generating a driving force of a vehicle such as an internal combustion engine or a driving motor, a driving force transmission mechanism for transmitting a driving force to wheels, a steering mechanism for adjusting a turning angle of a vehicle, and a control device such as a braking device that generates a braking force of a vehicle.

The body system control unit 12020 controls operations of various devices mounted in the vehicle body according to various programs. For example, the body system control unit 12020 serves as a control device of a keyless entry system, a smart key system, a power window device, or various lamps such as a head lamp, a back lamp, a brake lamp, a turn signal, and a fog lamp. In this case, radio waves transmitted from a portable device that substitutes for a key or signals of various switches can be input to the body system control unit 12020. The body system control unit 12020 receives inputs of these radio waves or signals and controls a door lock device, a power window device, a lamp, and the like of the vehicle.

The vehicle exterior information detection unit 12030 detects information outside the vehicle in which the vehicle control system 12000 is mounted. For example, an imaging unit 12031 is connected to the vehicle exterior information detection unit 12030. The vehicle exterior information detection unit 12030 causes the imaging unit 12031 to capture an image of the outside of the vehicle and receives the captured image. The vehicle exterior information detection unit 12030 may perform object detection processing or distance detection processing for peoples, cars, obstacles, signs, and letters on the road based on the received image.

The imaging unit 12031 is an optical sensor that receives light and outputs an electrical signal according to the amount of received light. The imaging unit 12031 can output an electrical signal as an image or output it as a distance measurement information. In addition, the light received by the imaging unit 12031 may be visible light or invisible light such as infrared light.

The vehicle interior information detection unit 12040 detects information on the inside of the vehicle. For example, a driver state detection unit 12041 that detects a driver's state is connected to the vehicle interior information detection unit 12040. The driver state detection unit 12041 includes, for example, a camera that captures an image of a driver, and the vehicle interior information detection unit 12040 may calculate a degree of fatigue or concentration of the driver or may determine whether or not the driver is dozing on the basis of detection information input from the driver state detection unit 12041.

The microcomputer 12051 can calculate a control target value of the driving force generator, the steering mechanism, or the braking device on the basis of the information on the inside and the outside of the vehicle acquired by the vehicle exterior information detection unit 12030 or the vehicle interior information detection unit 12040, and output a control command to the drive system control unit 12010. For example, the microcomputer 12051 can perform cooperative control aiming at realizing functions of advanced driver assistance system (ADAS) including vehicle collision avoidance or impact mitigation, follow-up traveling based on an inter-vehicle distance, vehicle speed maintenance traveling, vehicle collision warning, vehicle lane deviation warning, and the like.

Further, the microcomputer 12051 can perform coordinated control for the purpose of automated driving or the like in which autonomous travel is performed without depending on an operation of a driver by controlling the driving force generator, the steering mechanism, the braking device, and the like on the basis of information regarding the vicinity of the vehicle acquired by the vehicle exterior information detection unit 12030 or the vehicle interior information detection unit 12040.

In addition, the microcomputer 12051 can output a control command to the body system control unit 12020 based on the information outside the vehicle acquired by the vehicle exterior information detection unit 12030. For example, the microcomputer 12051 can perform cooperative control for antiglare such as switching a high beam to a low beam by controlling a headlamp according to a position of a preceding vehicle or an oncoming vehicle detected by the vehicle exterior information detection unit 12030.

The audio image output unit 12052 transmits an output signal of at least one of audio and an image to an output device capable of visually or audibly notifying an occupant of a vehicle or the outside of the vehicle of information. In the example illustrated in FIG. 20, an audio speaker 12061, a display unit 12062, and an instrument panel 12063 are illustrated as output devices. The display unit 12062 may include, for example, at least one of an onboard display and a head-up display.

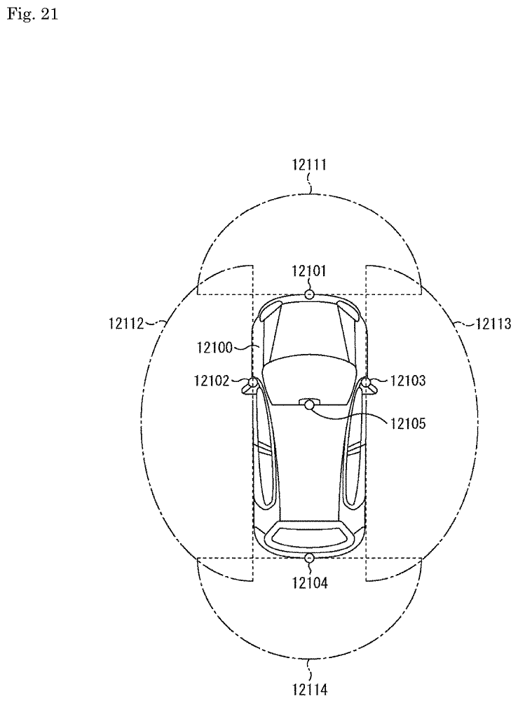

FIG. 21 is a diagram illustrating an example of positions at which the imaging unit 12031 is installed.

In FIG. 21, a vehicle 12100 includes imaging units 12101, 12102, 12103, 12104, and 12105 as the imaging unit 12031.

The imaging units 12101, 12102, 12103, 12104, and 12105 may be provided at positions such as a front nose, side-view mirrors, a rear bumper, a back door, and an upper part of a windshield in a vehicle interior of the vehicle 12100, for example. The imaging unit 12101 provided at the front nose and the imaging unit 12105 provided at an upper part of the windshield in a vehicle interior mainly obtain front view images of the vehicle 12100. The imaging units 12102 and 12103 provided on the side mirrors mainly acquire images on the lateral side of the vehicle 12100. The imaging unit 12104 provided on the rear bumper or the back door mainly acquires images in the rear of the vehicle 12100. The front view images acquired by the imaging units 12101 and 12105 are mainly used for detection of preceding vehicles, pedestrians, obstacles, traffic signals, traffic signs, lanes, and the like.

Note that FIG. 21 illustrates an example of imaging ranges of the imaging units 12101 to 12104. An imaging range 12111 indicates an imaging range of the imaging unit 12101 provided at the front nose, imaging ranges 12112 and 12113 respectively indicate the imaging ranges of the imaging units 12102 and 12103 provided at the side mirrors, and an imaging range 12114 indicates the imaging range of the imaging unit 12104 provided at the rear bumper or the back door. For example, a bird's-eye view image of the vehicle 12100 as viewed from above can be obtained by superimposition of image data captured by the imaging units 12101 to 12104.

At least one of the imaging units 12101 to 12104 may have a function for obtaining distance information. For example, at least one of the imaging units 12101 to 12104 may be a stereo camera constituted by a plurality of imaging elements or may be an imaging element having pixels for phase difference detection.

For example, the microcomputer 12051 can extract, particularly, a closest three-dimensional object on a path through which the vehicle 12100 is traveling, which is a three-dimensional object traveling at a predetermined speed (for example, 0 km/h or higher) in the substantially same direction as the vehicle 12100, as a preceding vehicle by acquiring a distance to each of three-dimensional objects in the imaging ranges 12111 to 12114 and temporal change in the distance (a relative speed with respect to the vehicle 12100) on the basis of distance information obtained from the imaging units 12101 to 12104. Further, the microcomputer 12051 can set an inter-vehicle distance which should be guaranteed in advance in front of a preceding vehicle and can perform automated brake control (also including following stop control) or automated acceleration control (also including following start control). In this way, it is possible to perform cooperated control in order to perform automated driving or the like in which a vehicle autonomously travels irrespective of an operation of a driver.

For example, the microcomputer 12051 can classify and extract three-dimensional object data regarding three-dimensional objects into two-wheeled vehicles, ordinary vehicles, large vehicles, pedestrians, and other three-dimensional objects such as utility poles on the basis of distance information obtained from the imaging units 12101 to 12104 and use the three-dimensional object data for automatic avoidance of obstacles. For example, the microcomputer 12051 identifies obstacles in the vicinity of the vehicle 12100 into obstacles that can be visually recognized by the driver of the vehicle 12100 and obstacles that are difficult to visually recognize. Then, the microcomputer 12051 can determine a risk of collision indicating the degree of risk of collision with each obstacle, and can perform driving assistance for collision avoidance by outputting a warning to a driver through the audio speaker 12061 or the display unit 12062 and performing forced deceleration or avoidance steering through the drive system control unit 12010 when the risk of collision has a value equal to or greater than a set value and there is a possibility of collision.

At least one of the imaging units 12101 to 12104 may be an infrared camera that detects infrared light. For example, the microcomputer 12051 can recognize a pedestrian by determining whether or not a pedestrian is present in images captured by the imaging units 12101 to 12104. Such recognition of a pedestrian is performed by, for example, a procedure of extracting a feature point in captured images of the imaging units 12101 to 12104 serving as infrared cameras, and a procedure of performing pattern matching processing on a series of feature points indicating the contour of an object to determine whether or not the object is a pedestrian. When the microcomputer 12051 determines that a pedestrian is present in the captured images of the imaging units 12101 to 12104 and recognizes the pedestrian, the audio image output unit 12052 controls the display unit 12062 such that a square contour line for emphasis is superimposed on the recognized pedestrian and is displayed. In addition, the audio image output unit 12052 may control the display unit 12062 so that an icon or the like indicating a pedestrian is displayed at a desired position.

In addition, for example, the technology according to the present disclosure may be applied to an endoscopic surgery system.

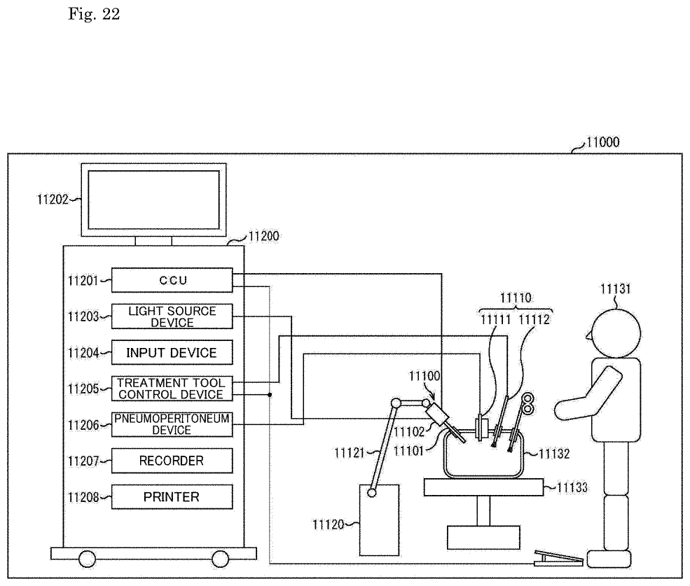

FIG. 22 is a diagram illustrating an example of a schematic configuration of an endoscopic surgery system to which the technology according to the present disclosure (the present technology) can be applied.

FIG. 22 illustrates a state where a surgeon (doctor) 11131 is performing a surgical operation on a patient 11132 on a patient bed 11133 by using the endoscopic surgery system 11000. As illustrated in the drawing, the endoscopic surgery system 11000 includes an endoscope 11100, other surgical instruments 11110 such as a pneumoperitoneum tube 11111 and an energized treatment tool 11112, a support arm device 11120 that supports the endoscope 11100, and a cart 11200 equipped with various devices for endoscopic operation.

The endoscope 11100 includes a lens barrel 11101, a region of which having a predetermined length from a distal end is inserted into a body cavity of the patient 11132, and a camera head 11102 connected to a proximal end of the lens barrel 11101. Although the endoscope 11100 configured as a so-called rigid mirror having the rigid lens barrel 11101 is illustrated in the illustrated example, the endoscope 11100 may be configured as a so-called flexible mirror having a flexible lens barrel.

An opening in which an objective lens is fitted is provided at the distal end of the lens barrel 11101. A light source device 11203 is connected to the endoscope 11100, and light generated by the light source device 11203 is guided to the distal end of the lens barrel by a light guide extending inside the lens barrel 11101 and is radiated toward the observation target in the body cavity of the patient 11132 via the objective lens. The endoscope 11100 may be a direct-viewing endoscope or may be a perspective endoscope or a side-viewing endoscope.

An optical system and an imaging element are provided inside the camera head 11102, and the reflected light (observation light) from the observation target is condensed on the imaging element by the optical system. The observation light is photoelectrically converted by the imaging element, and an electrical signal corresponding to the observation light, that is, an image signal corresponding to an observation image is generated. The image signal is transmitted as RAW data to a camera control unit (CCU) 11201.

The CCU 11201 is composed of a central processing unit (CPU), a graphics processing unit (GPU) or the like, and comprehensively controls the operation of the endoscope 11100 and a display device 11202. In addition, the CCU 11201 receives an image signal from the camera head 11102, and performs various types of image processing for displaying an image based on the image signal, for example, development processing (demosaic processing) on the image signal.

The display device 11202 displays an image based on an image signal having been subjected to image processing by the CCU 11201 under the control of the CCU 11201.

The light source device 11203 is constituted by, for example, a light source such as a light emitting diode (LED), and supplies irradiation light at the time of imaging a surgical part or the like to the endoscope 11100.

An input device 11204 is an input interface for the endoscopic surgery system 11000. The user can input various types of information or instructions to the endoscopic surgery system 11000 via the input device 11204. For example, the user inputs an instruction to change imaging conditions (a type of radiated light, a magnification, a focal length, or the like) of the endoscope 11100.

A treatment tool control device 11205 controls driving of the energized treatment tool 11112 for cauterizing or incising tissue, sealing a blood vessel, or the like. A pneumoperitoneum device 11206 sends gas into the body cavity through a pneumoperitoneum tube 11111 in order to inflate the body cavity of the patient 11132 for the purpose of securing a visual field for the endoscope 11100 and a working space for the surgeon. A recorder 11207 is a device capable of recording various information regarding operation. A printer 11208 is a device that can print various types of information regarding operation in various formats such as text, images, or graphs.

The light source device 11203 that supplies the endoscope 11100 with the radiation light for imaging the surgical part can be configured of, for example, an LED, a laser light source, or a white light source configured of a combination thereof. When a white light source is formed by a combination of RGB laser light sources, it is possible to control an output intensity and an output timing of each color (each wavelength) with high accuracy and thus, the light source device 11203 adjusts white balance of the captured image. Further, in this case, laser light from each of the respective RGB laser light sources is radiated to the observation target in a time division manner, and driving of the imaging element of the camera head 11102 is controlled in synchronization with radiation timing such that images corresponding to respective RGB can be captured in a time division manner. According to this method, it is possible to obtain a color image without providing a color filter to the imaging element.

Further, the driving of the light source device 11203 may be controlled to change the intensity of the output light at predetermined time intervals. It is possible to acquire images in a time-division manner by controlling the driving of the imaging element of the camera head 11102 in synchronization with a timing at which the intensity of the light is changed, and it is possible to generate a high dynamic range image without so-called blackout and whiteout by combining the images.

Further, the light source device 11203 may be configured to be able to supply light having a predetermined wavelength band corresponding to special light observation. In the special light observation, for example, by emitting light in a narrower band than irradiation light (that is, white light) during normal observation using wavelength dependence of light absorption in a body tissue, so-called narrow band light observation (narrow band imaging) in which a predetermined tissue such as a blood vessel in the mucous membrane surface layer is imaged with a high contrast is performed. Alternatively, in the special light observation, fluorescence observation in which an image is obtained by fluorescence generated by emitting excitation light may be performed. The fluorescence observation can be performed by emitting excitation light to a body tissue, and observing fluorescence from the body tissue (autofluorescence observation), or locally injecting a reagent such as indocyanine green (ICG) to a body tissue, and emitting excitation light corresponding to a fluorescence wavelength of the reagent to the body tissue to obtain a fluorescence image. The light source device 11203 can supply narrow band light and/or excitation light corresponding to such special light observation.

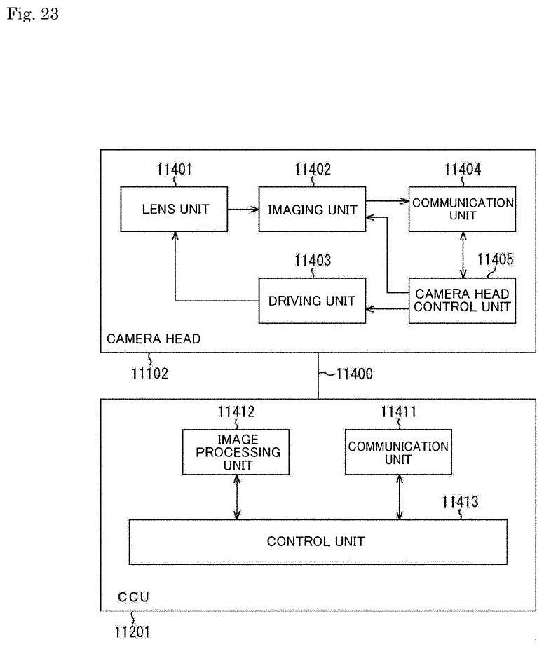

FIG. 23 is a block diagram illustrating an example of a functional configuration of the camera head 11102 and CCU 11201 illustrated in FIG. 22.

The camera head 11102 includes a lens unit 11401, an imaging unit 11402, a driving unit 11403, a communication unit 11404, and a camera head control unit 11405. The CCU 11201 includes a communication unit 11411, an image processing unit 11412, and a control unit 11413. The camera head 11102 and the CCU 11201 are connected to each other via a transmission cable 11400 so that they can communicate with each other.

The lens unit 11401 is an optical system provided at a portion for connection to the lens barrel 11101. The observation light taken in from the distal end of the lens barrel 11101 is guided to the camera head 11102 and incident on the lens unit 11401. The lens unit 11401 is configured in combination of a plurality of lenses including a zoom lens and a focus lens.

The imaging unit 11402 includes an imaging element. The imaging element constituting the imaging unit 11402 may be one element (so-called single plate type) or a plurality of elements (so-called multi-plate type). When the imaging unit 11402 is composed of a multi-plate type, for example, image signals corresponding to RGBs are generated by the imaging elements, and synthesized, and thereby a color image may be obtained. Alternatively, the imaging unit 11402 may include a pair of imaging elements for acquiring image signals for the right eye and the left eye corresponding to 3D (dimensional) display. When 3D display is performed, the surgeon 11131 can determine the depth of biological tissues in the surgical part more accurately. Here, when the imaging unit 11402 is composed of a multi-plate type, a plurality of lens units 11401 may be provided according to the imaging elements.

Further, the imaging unit 11402 may not necessarily be provided in the camera head 11102. For example, the imaging unit 11402 may be provided immediately after the objective lens inside the lens barrel 11101.

The driving unit 11403 includes an actuator, and moves the zoom lens and the focus lens of the lens unit 11401 by a predetermined distance along an optical axis under the control of the camera head control unit 11405. Accordingly, the magnification and focus of the image captured by the imaging unit 11402 can be adjusted appropriately.

The communication unit 11404 is configured of a communication device for transmitting or receiving various information to or from the CCU 11201. The communication unit 11404 transmits an image signal obtained from the imaging unit 11402 to the CCU 11201 through the transmission cable 11400 as RAW data.

The communication unit 11404 receives a control signal for controlling the driving of the camera head 11102 from the CCU 11201 and supplies the control signal to the camera head control unit 11405. The control signal includes, for example, information on the imaging conditions such as information indicating that the frame rate of the captured image is designated, information indicating that the exposure value at the time of imaging is designated, and/or information indicating that the magnification and the focus of the captured image are designated.

Note that the imaging conditions such as the frame rate, the exposure value, the magnification, and the focus described above may be appropriately designated by the user or may be automatically set by the control unit 11413 of the CCU 11201 on the basis of the acquired image signal. In the latter case, a so-called auto exposure (AE) function, auto focus (AF) function and auto white balance (AWB) function are provided in the endoscope 11100.

The camera head control unit 11405 controls the driving of the camera head 11102 on the basis of the control signal from the CCU 11201 received via the communication unit 11404.

The communication unit 11411 includes a communication device for transmitting/receiving various types of information to/from the camera head 11102. The communication unit 11411 receives an image signal transmitted from the camera head 11102 via the transmission cable 11400.

In addition, the communication unit 11411 transmits a control signal for controlling the driving of the camera head 11102 to the camera head 11102. The image signal or the control signal can be transmitted through electric communication, optical communication, or the like.

The image processing unit 11412 performs various kinds of image processing on the image signal that is the RAW data transmitted from the camera head 11102.

The control unit 11413 performs various kinds of control regarding imaging of the surgical part or the like using the endoscope 11100 and a display of a captured image obtained by imaging the surgical part or the like. For example, the control unit 11413 generates the control signal for controlling the driving of the camera head 11102.

Further, the control unit 11413 causes the display device 11202 to display the captured image obtained by imaging the surgical part or the like on the basis of the image signal having subjected to the image processing by the image processing unit 11412. In this case, the control unit 11413 may recognize various objects in the captured image using various image recognition technologies. For example, the control unit 11413 can recognize surgical tools such as forceps, specific biological parts, bleeding, mist when the energized treatment tool 11112 is used and the like by detecting the edge shape and color of the object included in the captured image. When the control unit 11413 causes the display device 11202 to display the captured image, it may cause various types of surgical support information to be superimposed and displayed with the image of the surgical part using the recognition result. When the surgical support information is superimposed and displayed, and presented to the surgeon 11131, it is possible to reduce the burden on the surgeon 11131 and the surgeon 11131 can reliably proceed the operation.

The transmission cable 11400 that connects the camera head 11102 to the CCU 11201 is an electrical signal cable compatible with communication of an electrical signal, an optical fiber compatible with optical communication, or a composite cable thereof.

Here, in the illustrated example, wired communication is performed using the transmission cable 11400, but the communication between the camera head 11102 and the CCU 11201 may be performed wirelessly.

Here, although the endoscopic surgery system has been described as an example, the technology according to the present disclosure may be applied to other, for example, a microscopic operation system.

Although the present disclosure has been described on the basis of the preferred examples, the present disclosure is not limited to these examples. The structures and configurations of the light receiving elements (the photoelectric conversion elements, the imaging elements), the light receiving devices, and the solid-state imaging devices, the manufacturing methods, and the used materials described in the examples are examples, and can be appropriately changed. The light receiving elements of the examples can be appropriately combined with each other. It is possible to capture a moving image and perform sensing using the solid-state imaging device based on the light receiving device of the present disclosure.

It is also possible to configure a light receiving device in which the light receiving element of the present disclosure and a light receiving element of the related art are mixed. That is, for example, the light receiving element unit in the present disclosure and a light receiving element unit that does not include a wire grid polarizing element may be alternately disposed. Such a light receiving device includes the light receiving element of the present disclosure, and thus it is possible to configure a light receiving device (solid-state imaging device) capable of acquiring polarization information at the same time in addition to performing normal imaging. That is, a polarization separation function for spatially performing polarization separation of polarization information of incident light can be given to the light receiving device (solid-state imaging device). Specifically, a light intensity, a polarization component intensity, and a polarization direction can be obtained in each light receiving element (imaging element), and thus, for example, it is possible to process image data on the basis of polarization information after imaging. For example, by applying desired processing to a portion of an image obtained by imaging the sky or window glass, a portion of an image obtained by imaging the water surface, or the like, it is possible to emphasize or reduce a polarization component or to separate various polarization components, and it is possible to improve the contrast of an image and delete unnecessary information. In addition, specifically, it is possible to perform such processing, for example, by specifying an imaging mode at the time of performing imaging using a solid-state imaging device. Further, it is possible to remove reflections on a windowpane by the solid-state imaging device and make boundaries (contours) of a plurality of objects clear by adding polarization information to image information. Alternatively, it is also possible to detect the condition of a road surface and detect obstacles on the road surface. Further, it is possible to perform imaging of a pattern reflecting the birefringence of an object, measurement of retardation distribution, acquisition of a polarizing microscope image, acquisition of a surface shape of an object, measurement of surface properties of an objects, detection of a moving object (a vehicle or the like), meteorological observation such as measurement of cloud distribution, and application to various fields. In addition, it is also possible to configure a solid-state imaging device that captures a three-dimensional image.

In some cases, it is possible to adopt a mode in which a groove portion (a type of element isolation region) having an insulating material or a light shielding material embedded therein and extending from a substrate to a lower side of a wire grid polarizing element is formed at an edge portion of a photoelectric conversion portion. Examples of the insulating material may include a material for forming an insulating film (insulating film formation layer) or an interlayer insulating layer, and examples of the light shielding material may include a material for forming the above-described light shielding film 24. By forming such a groove portion, it is possible to prevent a reduction in sensitivity, the occurrence of polarization crosstalk, and a reduction in an extinction ratio.

A waveguide structure may be provided between the photoelectric conversion portions 21. The waveguide structure is constituted by a thin film having a refractive index larger than a refractive index of a material for forming the lower layer and interlayer insulating layer 33, the thin film being formed in a region (for example, a cylindrical region) positioned between the photoelectric conversion portions 21 of the lower layer and interlayer insulating layer 33 (specifically, a portion of the lower layer and interlayer insulating layer 33) that covers the photoelectric conversion portion 21. Light incident from above the photoelectric conversion portion 21 is totally reflected by the thin film and reaches the photoelectric conversion portion 21. An orthogonal projection image of the photoelectric conversion portion 21 with respect to the semiconductor substrate 31 is positioned on the inner side of an orthogonal projection image of the thin film constituting the waveguide structure with respect to the semiconductor substrate 31. The orthogonal projection image of the photoelectric conversion portion 21 with respect to the semiconductor substrate 31 is surrounded by the orthogonal projection image of the thin film constituting the waveguide structure with respect to the substrate.

Alternatively, a light condensing pipe structure may be provided between the photoelectric conversion portions 21. The light condensing pipe structure is constituted by a light shielding thin film formed of a metal material or an alloy material, the thin film being formed in a region (for example, a cylindrical region) positioned between the photoelectric conversion portions 21 of the lower layer and interlayer insulating layer 33 covering the photoelectric conversion portions 21, and light incident from above the photoelectric conversion portion 21 is reflected by the thin film and reaches the photoelectric conversion portion 21. That is, an orthogonal projection image of the photoelectric conversion portion 21 with respect to the semiconductor substrate 31 is positioned on the inner side of an orthogonal projection image of the semiconductor substrate 31 of the thin film constituting the light condensing pipe structure. The orthogonal projection image of the photoelectric conversion portion 21 with respect to the semiconductor substrate 31 is surrounded by the orthogonal projection image of the semiconductor substrate 31 of the thin film constituting the light condensing pipe structure. The thin film can be obtained, for example, by forming the entire lower layer and interlayer insulating layer 33, forming a circular groove portion in the lower layer and interlayer insulating layer 33, and then filling the groove portion with a metal material or an alloy material.

It is possible to adopt a 2×2 pixel sharing method for sharing a selection transistor, a reset transistor, and an amplification transistor in 2×2 photoelectric conversion portions, and it is possible to provide a normal captured image obtained by performing imaging including polarization information in an imaging mode in which pixel addition is not performed, and integrating all polarization components in a mode in which FD-addition is performed on accumulated charge in a 2×2 sub-pixel region.

Further, in the examples, a case where a CMOS type solid-state imaging device configured such that unit pixels detecting signal charge corresponding to the amount of incident light as a physical amount are disposed in a matrix is applied has been described as an example, but the present disclosure is not limited to the application to the CMOS type solid-state imaging device and can also be applied to a CCD type solid-state imaging device. In the latter case, the signal charge is transferred in a vertical direction by a vertical transfer register having a CCD type structure and is transferred in a horizontal direction by a horizontal transfer register, and a pixel signal (image signal) is output by the amplification of the signal charge. In addition, the present disclosure is not limited to all column type solid-state imaging devices in which pixels are formed in a two-dimensional matrix, and column signal processing circuits are disposed for each pixel column. Further, in some cases, a selection transistor can also be omitted.

Further, the light receiving element (imaging element) of the present disclosure is not limited to the application to a solid-state imaging device that detects a distribution of the amount of incident light of visible light and captures the distribution as an image, and can also be applied to a solid-state imaging device that captures a distribution of the amount of incident light of infrared light, X-rays, particles, or the like as an image. Further, in a broad sense, the present disclosure can be applied to all solid-state imaging devices (physical amount distribution detection devices) such as a fingerprint detection sensor that detects distributions of other physical amounts such as pressure and capacitance and captures the distributions as images.

Further, the present disclosure is not limited to a solid-state imaging device that scans unit pixels in an imaging region in order in units of rows and reads out pixel signals from the unit pixels. The present disclosure can also be applied to an X-Y address type solid-state imaging device that selects any pixels in units of pixels and reads out pixel signals in units of pixels from the selected pixels. A solid-state imaging device may be formed as an one chip, or may be configured to have a module shape having an imaging function in which an imaging region and a drive circuit or an optical system are packaged together.

In addition, the present disclosure is not limited to the application to a solid-state imaging device and can also be applied to an imaging device. Here, the imaging device indicates a camera system such as a digital still camera or a video camera, and electronic equipment having an imaging function such as a mobile phone. A module type mounted in electronic equipment, that is, a camera module may also be used as an imaging device.

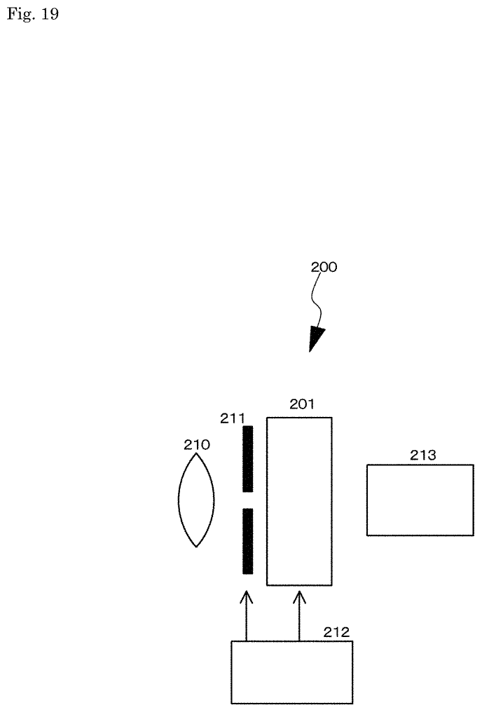

An example in which a solid-state imaging device 201 of the present disclosure is used in electronic equipment (camera) 200 is illustrated as a conceptual diagram in FIG. 19. The electronic equipment 200 includes the solid-state imaging device 201, an optical lens 210, a shutter device 211, a drive circuit 212, and a signal processing circuit 213. The optical lens 210 forms an image of image light (incident light) from a subject on an imaging surface of the solid-state imaging device 201. Thus, signal charges are accumulated in the solid-state imaging device 201 for a certain period of time. The shutter device 211 controls a light irradiation period and a light blocking period for the solid-state imaging device 201. The drive circuit 212 supplies a drive signal for controlling a transfer operation of the solid-state imaging device 201 and a shutter operation of the shutter device 211. Signal transfer of the solid-state imaging device 201 is performed by the drive signal (timing signal) supplied from the drive circuit 212. The signal processing circuit 213 performs various signal processing. A video signal that has undergone signal processing is stored in a storage medium such as a memory, or is output to a monitor. In such electronic equipment 200, miniaturization of a pixel size of the solid-state imaging device 201 and an improvement in transfer efficiency can be achieved, and thus it is possible to obtain the electronic equipment 200 with improved pixel characteristics. The electronic equipment 200 to which the solid-state imaging device 201 is applicable is not limited to a camera, and can also be applied to imaging devices such as a digital still camera and a camera module for mobile equipment such as a mobile phone.

It is possible to detect, for example, the taste and freshness of food on the basis of the light receiving device of the present disclosure. For example, a peak wavelength of a detection band in a case where myoglobin indicating a delicious component of tuna, beef, or the like is detected is in the range of 580 nm to 630 nm, and a half-value width is in the range of 30 nm to 50 nm. A peak wavelength of a detection band in a case where oleic acid indicating freshness of tuna, beef, or the like is detected is 980 nm, and a half-value width is in the range of 50 nm to 100 nm. A peak wavelength of a detection band in a case where chlorophyll indicating freshness of leaf vegetables such as Brassica rapa is detected is in the range of 650 nm to 700 nm, and a half-value width is in the range of 50 nm to 100 nm.

In addition, it is possible to detect sugar content and water content of fruits on the basis of the light receiving device of the present disclosure.

For example, a peak wavelength of a detection band in a case where a flesh optical path length indicating the sugar content of Raiden, which is a kind of melon, is detected is 880 nm, and a half-value width is in the range of 20 nm to 30 nm. A peak wavelength of a detection band in a case where sucrose indicating the sugar content of Raiden is detected is 910 nm, and a half-value width is in the range of 40 nm to 50 nm. A peak wavelength of a detection band in a case where sucrose indicating the sugar content of Raiden Red, which is another kind of melon, is detected is 915 nm, and a half-value width is in the range of 40 nm to 50 nm. A peak wavelength of a detection band in a case where water content indicating the sugar content of Raiden Red is detected is 955 nm, and a half-value width is in the range of 20 nm to 30 nm.

A peak wavelength of a detection band in a case where sucrose indicating the sugar content of apples is detected is 912 nm, and a half-value width is in the range of 40 nm to 50 nm. A peak wavelength of a detection band in a case where water indicating the water content of mandarin orange is detected is 844 nm, and a half-value width is 30 nm. A peak wavelength of a detection band in a case where sucrose indicating the sugar content of mandarin orange is detected is 914 nm, and a half-value width is in the range of 40 nm to 50 nm.

In addition, it is possible to classify plastics on the basis of the light receiving device of the present disclosure. For example, a peak wavelength of a detection band in a case where polyethylene terephthalate (PET) is detected is 1669 nm, and a half-value width is in the range of 30 nm to 50 nm. A peak wavelength of a detection band in a case where polystyrene (PS) is detected is 1688 nm, and a half-value width is in the range of 30 nm to 50 nm. A peak wavelength of a detection band in a case where polyethylene (PE) is detected is 1735 nm, and a half-value width is in the range of 30 nm to 50 nm. A peak wavelength of a detection band in a case where polyvinyl chloride (PVC) is detected is in the range of 1716 nm to 1726 nm, and a half-value width is in the range of 30 nm to 50 nm. A peak wavelength of a detection band in a case where polypropylene (PP) is detected is in the range of 1716 nm to 1735 nm, and a half-value width is in the range of 30 nm to 50 nm.

In addition, the light receiving device of the present disclosure can be applied to freshness management of cut flowers.

Further, the light receiving device of the present disclosure can be applied to inspection of foreign substances mixed in food. The light receiving device of the present disclosure can be applied to, for example, the detection of foreign substances such as skins, shells, stones, leaves, branches or wood chips mixed in nuts and fruits such as almonds, blueberries, and walnuts. In addition, the light receiving device of the present disclosure can be applied to, for example, the detection of foreign substances such as plastic pieces mixed in processed foods, beverages, or the like. Alternatively, the light receiving device of the present disclosure can be applied to the detection of a normalized difference vegetation index (NDVI) which is an index of vegetation.

In addition, the light receiving device of the present disclosure can be applied to, for example, the detection of a person based on either one or both of a spectral shape with a wavelength of approximately 580 nm derived from hemoglobin of human skin and a spectral shape with a wavelength of approximately 960 nm derived from a melanin pigment contained in human skin.

Further, the light receiving device of the present disclosure can be applied to, for example, biometric detection (biometric authentication), forgery prevention and monitoring of a user interface and a signature, and the like.

In addition, the present disclosure can also adopt the following configurations.

[A01]<<Light Receiving Element>>

A light receiving element including:

a wire grid polarizing element, wavelength selection means, and a photoelectric conversion portion from a light incident side,

wherein the wavelength selection means includes a plurality of wavelength selection members, and

the wavelength selection members constituted by a plasmon filter transmit light having different wavelengths.

[A02] The light receiving element according to [A01],

wherein each wavelength selection member is constituted by a periodic structure, and

the periodic structure includes a substrate and a plurality of holes provided in the substrate.

[A03] The light receiving element according to [A02],

wherein when a light absorption axis of the wire grid polarizing element is set to be a first direction, and a light transmission axis of the wire grid polarizing element is set to be a second direction, an arrangement direction of the plurality of holes is parallel to the second direction.

[A04] The light receiving element according to [A03],

wherein a hole group is constituted by the plurality of holes arranged in the second direction and constituting each wavelength selection member, and a plurality of hole groups are arranged in the first direction.

[A05] The light receiving element according to [A04],

wherein the hole groups constituting each wavelength selection member are alternately arranged in the first direction.

[A06] The light receiving element according to [A01],

wherein each wavelength selection member is constituted by a periodic structure, and

the periodic structure includes a base layer, dots provided on the base layer, and a dielectric material provided on the base layer and is filled between the dots.

[A07] The light receiving element according to [A06],

wherein when a light absorption axis of the wire grid polarizing element is set to be a first direction, and a light transmission axis of the wire grid polarizing element is set to be a second direction, an arrangement direction of the plurality of dots is parallel to the second direction.

[A08] The light receiving element according to [A07],

wherein a dot group is constituted by the plurality of dots arranged in the second direction and constituting each wavelength selection member, and a plurality of dot groups are arranged in the first direction.

[A09] The light receiving element according to [A08],

wherein the dot groups constituting each wavelength selection member are alternately arranged in the first direction.

[A10] The light receiving element according to [A01],

wherein each wavelength selection member is constituted by a periodic structure, and

the periodic structure includes a base layer and a plurality of belt-like conductive material layers provided on the base layer.

[A11] The light receiving element according to [A10],

wherein when a light absorption axis of the wire grid polarizing element is set to be a first direction, and a light transmission axis of the wire grid polarizing element is set to be a second direction, a repetition direction of the belt-like conductive material layers is the second direction.

[A12] The light receiving element according to [A11],

wherein a conductive material layer group is constituted by the plurality of belt-like conductive material layers arranged in the second direction and constituting each wavelength selection member, and a plurality of conductive material layer groups are arranged in the first direction.

[A13] The light receiving element according to [A12],

wherein the conductive material layer groups constituting each wavelength selection member are alternately arranged in the first direction.

[B01]<<a Light Receiving Device>>

A light receiving device including:

a plurality of light receiving element units each of which is constituted by a plurality of light receiving elements,

wherein each of the light receiving elements constituting each light receiving element unit includes a wire grid polarizing element, wavelength selection means, and a photoelectric conversion portion from a light incident side,

the wavelength selection means includes a plurality of wavelength selection members, and

the wavelength selection members constituted by a plasmon filter transmit light having different wavelengths.

[B02] The light receiving device according to [B01],

wherein the light receiving element unit includes four light receiving elements arranged in a 2×2 array,

a polarization direction of transmission performed by the wire grid polarizing element constituting a first light receiving element is at α degrees,

a polarization direction of transmission performed by the wire grid polarizing element constituting a second light receiving element is at (α+45) degrees,

a polarization direction of transmission performed by the wire grid polarizing element constituting a third light receiving element is at (α+90) degrees, and

a polarization direction of transmission performed by the wire grid polarizing element constituting a fourth light receiving element is at (α+135) degrees.

### REFERENCE SIGNS LIST

10A, 10A₁, 10A₂, 10A₃, 10A₄ Light receiving element unit
10B Light receiving element group
11 Light receiving element
21 Photoelectric conversion portion
22 Gate portion constituting memory portion
23 High-concentration impurity region constituting memory portion
24 Light shielding film
31 Silicon semiconductor substrate
32 Wiring layer
33 Lower layer and interlayer insulating layer
34 Base insulating layer
35 Interlayer film
36 Upper layer and interlayer insulating layer
50, 501, 502, 503, 504 Wire grid polarizing element
51A Light reflecting layer formation layer
52 Insulating film
52A Insulating film formation layer
52a Notch portion of insulating film
53 Light absorbing layer
53A Light absorbing layer formation layer
54 Line portion (laminated structure)
55 Space portion (gap between laminated structures)
56 Protection film
57 Second protection film
58 Third protection film
59 Frame portion
60, 601, 602, 603, 604 Wavelength selection means
60′ Wavelength selection means and extended portion
60a Substrate
61, 64, 67 Wavelength selection member
62 Hole group
63 Hole
65 Dot group
66 Dot
68 Conductive material layer group
69 Conductive material layer
81 On-chip microlens
100 Solid-state imaging device
101 Photoelectric conversion portion (imaging element)
111 Imaging region (effective pixel region)
112 Vertical drive circuit
113 Column signal processing circuit
114 Horizontal drive circuit
115 Output circuit
116 Driving control circuit
117 Signal line (data output line)
118 Horizontal signal line
200 Electronic equipment (camera)
201 Solid-state imaging device
210 Optical lens
211 Shutter device
212 Drive circuit
213 Signal processing circuit
FD Floating diffusion layer
TRₘₑₘ Memory portion
TRₜrs Transfer transistor
TRᵣₛₜ Reset transistor
TRₐₘₚ Amplification transistor
TRₛₑₗ Selection transistor
V_{DD} Power supply
MEM Memory selection line
TG Transfer gate line
RST Reset line
SEL Selection line
VSL Signal line (data output line)

What is claimed is:

1. A light receiving element, comprising:
a wire grid polarizing element, wavelength selection means, and a photoelectric conversion portion from a light incident side,
wherein the wavelength selection means includes a plurality of wavelength selection members, and
the wavelength selection members constituted by a plasmon filter transmit light having different wavelengths.

2. The light receiving element according to claim 1,
wherein each wavelength selection member is constituted by a periodic structure, and
the periodic structure includes a substrate and a plurality of holes provided in the substrate.

3. The light receiving element according to claim 2,
wherein when a light absorption axis of the wire grid polarizing element is set to be a first direction, and a light transmission axis of the wire grid polarizing element is set to be a second direction, an arrangement direction of the plurality of holes is parallel to the second direction.

4. The light receiving element according to claim 3,
wherein a hole group is constituted by the plurality of holes arranged in the second direction and constituting each wavelength selection member, and a plurality of hole groups are arranged in the first direction.

5. The light receiving element according to claim 4,
wherein the hole groups constituting each wavelength selection member are alternately arranged in the first direction.

6. The light receiving element according to claim 1,
wherein each wavelength selection member is constituted by a periodic structure, and
the periodic structure includes a base layer, dots provided on the base layer, and a dielectric material provided on the base layer and is filled between the dots.

7. The light receiving element according to claim 6,
wherein when a light absorption axis of the wire grid polarizing element is set to be a first direction, and a light transmission axis of the wire grid polarizing element is set to be a second direction, an arrangement direction of the plurality of dots is parallel to the second direction.

8. The light receiving element according to claim 7,
wherein a dot group is constituted by the plurality of dots arranged in the second direction and constituting each wavelength selection member, and a plurality of dot groups are arranged in the first direction.

9. The light receiving element according to claim 8,
wherein the dot groups constituting each wavelength selection member are alternately arranged in the first direction.

10. The light receiving element according to claim 1,
wherein each wavelength selection member is constituted by a periodic structure, and
the periodic structure includes a base layer and a plurality of belt-like conductive material layers provided on the base layer.

11. The light receiving element according to claim 10,
wherein when a light absorption axis of the wire grid polarizing element is set to be a first direction, and a light transmission axis of the wire grid polarizing element is set to be a second direction, a repetition direction of the belt-like conductive material layers is the second direction.

12. The light receiving element according to claim 11.
wherein a conductive material layer group is constituted by the plurality of belt-like conductive material layers arranged in the second direction and constituting each wavelength selection member, and a plurality of conductive material layer groups are arranged in the first direction.

13. The light receiving element according to claim 12,
wherein the conductive material layer groups constituting each wavelength selection member are alternately arranged in the first direction.

14. A light receiving device, comprising:
a plurality of light receiving element units each of which is constituted by a plurality of light receiving elements,
wherein each of the light receiving elements constituting each light receiving element unit includes a wire grid polarizing element, wavelength selection means, and a photoelectric conversion portion from a light incident side,
the wavelength selection means includes a plurality of wavelength selection members, and
the wavelength selection members constituted by a plasmon filter transmit light having different wavelengths.

15. The light receiving device according to claim 14,
wherein the light receiving element unit includes four light receiving elements arranged in a 2×2 array,
a polarization direction of transmission performed by the wire grid polarizing element constituting a first light receiving element is at α degrees,
a polarization direction of transmission performed by the wire grid polarizing element constituting a second light receiving element is at (α+45) degrees,
a polarization direction of transmission performed by the wire grid polarizing element constituting a third light receiving element is at (α+90) degrees, and
a polarization direction of transmission performed by the wire grid polarizing element constituting a fourth light receiving element is at (α+135) degrees.
